(19) United States
(12) Patent Application Publication
(10) Pub. No.: US 2009/0232915 A1
(43) Pub. Date: Sep. 17, 2009

(54) MIXTURES WITH A COLLAGEN SYNTHESIS BOOSTING ACTION

(75) Inventors: Gerhard Schmaus, Hoxter-Bosseborn (DE); Helge Franke, Dieburg (DE); Sabine Lange, Holzminden (DE)

Correspondence Address: CONNOLLY BOVE LODGE & HUTZ LLP, 1875 EYE STREET, N.W., SUITE 1100, WASHINGTON, DC, 20006, US

(73) Assignee: SYMRISE GmbH & Co., KG, Holzminden (DE)

(21) Appl. No.: 12/401,390

(22) Filed: Mar. 10, 2009

Related U.S. Application Data
(60) Provisional application No. 61/036,622, filed on Mar. 14, 2008.

(51) Int. Cl. A61K 8/97; A61Q 19/08

(52) U.S. Cl. 424/744

(57) ABSTRACT

The invention relates to synergistically active, ternary mixtures which boost collagen synthesis and to the uses thereof, in particular for slowing down skin ageing and for wound healing of damaged skin. The invention furthermore relates to the use of the ternary mixtures according to the invention for treating the oral cavity and pharynx, and in particular therein for preventing and slowing down periodontitis, for building up periodontal connective tissue and for preventing and treating infections in the oral cavity and for wound healing in the oral cavity.

The invention relates to the field of synergistically active, ternary mixtures which boost collagen synthesis and to the uses thereof in particular for cosmetic, oral hygiene and pharmaceutical purposes. Specifically, the invention relates to the production of ternary mixtures containing (a) at least one extract of Aloe and (b) at least one substance selected from the group ascorbic acid and the derivatives thereof and (c) at least one extract of a rosaceous plant (Rosaceae, rose family), preferably raspberry leaf extract, strawberry leaf extract, blackberry leaf extract or meadowsweet herb extract. The invention furthermore relates to the use of the ternary mixtures for stimulating collagen synthesis, in particular for the purpose of slowing down skin ageing, for reducing wrinkle formation, for treating inflammatory processes of the skin and for wound healing of damaged skin. The invention furthermore relates to the use of the ternary mixtures according to the invention for treating the oral cavity and pharynx, in particular for preventing and slowing down periodontitis and for building up periodontal connective tissue and moreover for preventing and treating infections in the oral cavity and for wound healing in the oral cavity.

Ageing of human skin is accompanied by increasing wrinkle formation and declining elasticity and strength. In the context of the ageing process, a distinction is drawn between intrinsic and extrinsic skin ageing. Intrinsic ageing encompasses the natural, genetically determined changes in the skin. Extrinsic skin ageing describes premature ageing processes which are brought about by exogenous influences such as sunlight, environmental poisons (e.g. ozone, tobacco smoke etc.), psychological stress and chronic inflammation. Ultraviolet radiation is the most significant exogenous noxious agent to cause premature ageing of human skin (photoageing). In addition to natural sunlight, irradiation of the human skin with artificial UV radiation (solarium) is becoming ever more significant.

The structural changes responsible for the clinical picture of aged skin primarily take place in the dermis. The elasticity and strength of skin are substantially determined by the two main constituents of the dermal extracellular matrix, the two fibrous proteins collagen and elastin. The dermis mainly contains collagen 1 (90-85%), which is formed exclusively by dermal fibroblasts, and significantly smaller amounts (10-15%) of collagen 3. Elastin, the main constituent of the elastic fibers of the skin in addition to fibrillin, is present in the dermis in an amount of approx. 1-3%.

In comparison with young skin, old skin is characterized by a declining concentration of collagen and elastin. This age-related tissue loss is brought about by the reduction in de novo collagen synthesis which accompanies ageing and by an imbalance between the activation and inhibition of proteolytic activity, which is accompanied by increased degradation of collagen.

Offsetting the effects brought about by intrinsic and extrinsic skin ageing such as reduced de novo collagen synthesis and increased proteolytic activity by MMPs, in particular MMP-1, -2, -3 and -9, is thus an important goal in the development of new cosmetic active ingredients to counter skin ageing and wrinkles.

Retaining an elevated collagen content in the skin or increasing the collagen content in the skin may be achieved in various different ways. On the one hand, substances which inhibit matrix metalloproteinases may be used. On the other hand, however, it is also possible to use substances which increase collagen synthesis in order, by de novo synthesis, to counter the negative effects of MMP-induced collagen degradation. The reduction in de novo collagen synthesis which accompanies increasing age may here be at least partially compensated by using active ingredients which increase collagen synthesis.

Individual substances which are frequently mentioned in connection with increasing collagen synthesis and are thus prior art are for example active ingredients such as ascorbic acid and the derivatives thereof, retinol and derivatives of retinol or plant extracts such as for example extracts of Aloe and Centella species. Active ingredients which are furthermore frequently used to boost collagen synthesis also include peptide substances and the derivatives thereof such as e.g. carnitine, carnosine, creatine, matrikine peptides (e.g. lysyl-threonyl-threonyl-lysyl-serine) and further peptide structures such as palmitoylated pentapeptides (e.g. Matrixyl from Sederma) or the oligopeptide with the trade name Vincipeptide (from Vincience, France). Moreover, compounds such as asiatic acid, madecassic acid, madecassoside, asiaticoside, extracts of Centella asiatica, niacinamide, astaxanthine, glucans e.g. from yeasts and oats, soy extract and soy isoflavones, such as genistein and daidzein, rutin, chrysin, morin, betel nut alkaloids, forskolin, betulinic acid, extracts of Plantago species, TGF-beta, extracts of Ginkgo biloba, glutamine and glycolic acid are used as collagen synthesis stimulators. In contrast, no mention has been made of achieving a synergistically enhanced boost in collagen synthesis by the use of the ternary mixtures according to the invention.

Matrix metalloproteinases, a group of enzymes which are capable of proteolytically degrading the macromolecules of the extracellular matrix (ECM), which also include collagen, also play a significant role in skin ageing. Ultimately, this means that MMPs precisely counteract de novo collagen synthesis. It has accordingly been established that old skin has a content of MMPs which is distinctly higher than that of young skin (J. H. Chung et al., J. Invest. Dermatol, 2001, 117, 1218-1224). MMPs have broad, often overlapping substrate specificity and, in combination, they are capable of breaking down all the protein components of the extracellular matrix. Around 20 MMPs have hitherto been identified. The are generally secreted as inactive proenzymes (pro-MMP). In human skin, a major role is played primarily by MMP-1 (collagenase 1), MMP-2 (gelatinase A), MMP-9 (gelatinase B) and MMP-3. Apart from cleaving collagen 1 and 3, MMP-1 also cleaves pro-MMP-2 and pro-MMP-9, so activating them. MMP-2 and MMP-9 are among the elastin-degrading proteases (A. Thibodeau, Cosmetics & Toiletries 2000, 115 (11), 75-82).

MMPs also play a decisive role in premature skin aging brought about by exogenous factors. A still further increased level of MMPs has been detected in light-aged skin relative to aged skin provided with light protection (J. H. Chung et al., J. Invest. Dermatol, 2001, 117, 1218-1224). Induction of matrix metalloproteinases has been demonstrated not only for UVA and UVB radiation, but also for infrared radiation. Such induction has been observed both in vitro in cultured human dermal fibroblasts and in vivo in UV-irradiated human skin. Stimulation with tobacco smoke also led in human dermal fibroblasts to upregulation of MMP-1 and -3 expression (J. Krutmann, Hautarzt 2003, 54, 809-817).

The use of substances which inhibit MMP-1 (e.g. retinyl palmitate, propyl gallate, precocene, 6-hydroxy-7-methoxy-2,2-dimethyl-1(2H)-benzopyran, 3,4-dihydro-6-hydroxy-7-methoxy-2,2-dimethyl-1 (2H)-benzopyran) for preventing sunlight- and/or heat-induced ageing of human skin is known and has been described e.g. in WO 01/74320.

It is furthermore known that matrix metalloproteinases are in particular also of significance in pathological changes in the oral cavity, e.g. of the periodontium. Periodontitis (also known as “periodontosis”) is an inflammation of the periodontium, i.e. of the tooth supporting structures. The periodontium comprises various tissues: the gum epithelium (gingiva), the connective tissue of the gingiva, the periodontal ligament (desmodontium), the root cement and the surrounding alveolar bone. The desmodontium is located between the root surface and the alveolar bone. This is a cell-rich connective tissue which anchors the tooth in the bony tooth socket, the alveolus. The desmodontal gap is 53-74% occupied by collagen and oxytalan fiber bundles. That part of the desmodontal fibers embedded in the root cement and in the alveolar bone anchors the tooth in the alveolus. Among the main clinical signs of periodontitis are inflammation of the gums associated with wound formation, loss of attachment, formation of periodontal pockets and alveolar bone breakdown.

The main cause of periodontitis is plaque. This consists of certain constituents of the saliva, food residues and, above all, of bacteria and their breakdown products. This particular type of infectious disease is in most cases caused by Porphyromonas gingivalis, Bacteroides forsythus and Actinobacillus actinomycetemcomitans. Continuous release of bacterial toxins, in particular of lipopolysaccharides, probably triggers the release of proinflammatory mediators, such as e.g. IL-1beta, TNF-alpha and PGE2 in the patients affected tissues. These signaling substances stimulate the infiltration of immunocompetent cells into the colonized tissue. Immigration of neutrophilic granulocytes and macrophages subsequently leads to inflammation of the gums (gingivitis) and to the release of proinflammatory mediators such as for example IL-1 and IL-6. These in turn activate the synthesis in the skin and mucous membranes of matrix-degrading metalloproteinases (matrix metalloproteinases, MMPs), which break down the extracellular matrix of the surrounding connective tissue. As a result, bacteria, which initially interacted with the gingival line, penetrate deep into the underlying connective tissue, where they continue inflammatory processes and the synthesis of MMPs and ultimately detach the uppermost layer of the epithelium from the dental root. This results in the formation of a periodontal pocket. The body's response involves inflammation of the gingiva and of the periodontium accompanied by damage to the alveolar bone. In the final stage of periodontitis, the sufferer is at risk of massive tooth loss, furthermore accompanied by infections of the oral cavity and wound formation.

Studies (T. Kuboto et al., Arch. Oral. Biol. 1996, 41, 253-262; A. L. Ejeil et al., J. Periodontol. 2003, 74, 188-195) have shown that the levels of a series of matrix metalloproteinases (MMP-1, -3, -8, -9 and -13) are significantly increased in patients with inflammation of the gums in comparison with patients with healthy gums. These levels correlate with the severity of the gingivitis or periodontitis. There is furthermore a significant decrease in collagen fibers as the extent of inflammation of the gums increases. MMP-9 here clearly acts as a marker in the early stage of periodontitis (A. L. Ejeil et al., J. Periodontol. 2003, 74, 188-195). Compensating these effects caused by the boost in collagen synthesis with the assistance of the synergistically active ternary mixtures is therefore likewise advantageously indicated.

MMPs also play an important part in the genesis of caries and in losses of hard tooth structure, such as for example erosion, which are not caused by caries. Teeth are mainly composed of a bone-like substance known as dentine. In the area of the dental crown which projects out from the gums, the dentine is coated with a protective layer of dental enamel. Approx. 30% of the dentine consists of a cell-free matrix primarily containing glycoproteins, in which are embedded collagen fibers and inorganic constituents.

The genesis of caries and erosion accompanies demineralization of the teeth. Mineral substances are largely responsible for tooth hardness. The formation of acids by oral bacteria after consuming sugary foodstuffs, on the one hand, as well as frequent contact with strongly acidic beverages (e.g. fruit juices) and strongly acidic foods (tropical fruit, pineapple etc.) result in demineralization of the dental enamel and, as the process progresses, also of the dentine. Demineralized dentine is susceptible to degradation. It has been possible to demonstrate in vitro that degradation of the organic matrix is necessary for the formation of a hole in the tooth. Tjäderhane et al. (J. Dent. Res. 1998, 77, 1622-1629) detected MMP-2, MMP-8 and MMP-9 in caries lesions and established that these are activated by acids. Slowing down and ideally completely suppressing these effects largely brought about by collagen degradation with the assistance of the synergistic active ternary mixtures is therefore likewise advantageously indicated.

Pashley et al. (J. Dent. Res. 2004, 83, 216-221) have shown that, even in the absence of bacteria, collagenolytically active proteinases cause degradation of collagen fibers of the organic matrix in dentine which has been partially demineralized by acids. Degradation of collagen fibers was prevented by addition of chlorhexidine or protease inhibitors (MMP inhibitor: benzamidine hydrochloride, cysteine protease inhibitor: N-ethylmaleimide, epsilon-amino-n-caproic acid, serine protease inhibitor: phenylmethylsulfonyl fluoride). Slowing down and ideally completely suppressing these effects largely brought about by collagen degradation with the assistance of the synergistic active ternary mixtures is therefore likewise advantageously indicated.

Maintaining the health or slowing the degradation of the connective tissue of the periodontium and the collagen fibers of the teeth, on the one hand, by preventing damage by MMPs by using matrix metalloproteinase inhibitors and, on the other hand, by increased formation of collagen by collagen synthesis boosting active ingredients are therefore important strategies when developing new active ingredients for oral care or oral hygiene. If the described processes are to be effectively halted, the damage caused by MMPs and in this case in particular by MMP-1 (collagenase 1), MMP-2 (gelatinase A), MMP-8 (collagenase 2) and MMP-9 (gelatinase B) must be inhibited at a very early stage.

Various publications describe the use of synthetic MMP inhibitors in periodontal disease (M. E. Ryan et al., Curr. Opin. Periodontal. 1996, 3, 85-96; R. Gendron et al., Clin. Diagn. Lab. Immunol. 1999, 6, 437-439). Compensating these effects by boosting the formation of collagen with the assistance of the synergistically active ternary mixtures is likewise advantageously indicated, since substances which boost collagen synthesis counteract by de novo synthesis the negative effects of MMP-induced collagen degradation.

It was accordingly the object of the present invention to provide agents which significantly boost de novo collagen synthesis relative to the prior art and so contribute to slowing down skin ageing and to protecting the skin and mucous membranes of the periodontium and to protecting the organic matrix of the dentine or counteracting damage to and/or excessive degradation of collagen. The agents according to the invention should, insofar as use on a user's skin is desired, where possible be particularly active against extrinsic skin ageing and the exogenous influencing factors associated therewith. The agents to be provided should, where possible, be of natural origin, be easy to manufacture, have good storage properties and be usable in numerous different preparations, in particular in cosmetic and pharmaceutical preparations and in preparations for oral hygiene. It is furthermore intended to provide production methods for corresponding agents and uses thereof.

For the purposes of the invention, an oral hygiene product (hereinafter also referred to as an oral care product or oral hygiene preparation) is taken to mean one of the formulations familiar to a person skilled in the art for cleaning and caring for the oral and pharyngeal cavity and for freshening the breath. This expressly includes care of the teeth and gums. Dosage forms of customary oral hygiene formulations are creams, gels, pastes, foams, emulsions, suspensions, aerosols, sprays and capsules, granules, pastilles, tablets, candies or chewing gums, this list being understood not to be limiting for the purposes of the present invention.

The present invention accordingly provides a ternary mixture containing, substantially consisting of or consisting of

a) an extract of Aloe plant material,
b) ascorbic acid and/or the derivative(s) thereof, preferably at least one solvate and/or salt of ascorbic acid and/or at least one ascorbic acid prodrug, and
c) an extract of rosaceous plant material,
except for an aftersun balm simultaneously containing 3.0 wt. % of an Aloe barbadensis leaf extract 10/1 in water, 1.0 wt. % of sodium ascorbyl phosphate and 0.5 wt. % of maltodextrin Rubus fruticosus leaf extract, in each case relative to the entire balsam.

Cosmetic and pharmaceutical agents which boost collagen synthesis are already known as such. Agents which are already frequently used for this purpose are retinol derivatives such as retinoic acid, retinal, retinol, retinyl acetate or retinyl palmitate, ascorbic acid and the derivatives thereof such as in particular ascorbyl palmitate, magnesium ascorbyl phosphate, sodium ascorbyl phosphate and ascorbyl alpha- and beta-glucoside. In addition, a whole series of further active ingredients and plant extracts are described as stimulators of collagen synthesis: peptide substances and the derivatives thereof such as e.g. carnitine, carnosine, creatine, matrikine peptides (e.g. lysyl-threonyl-threonyl-lysyl-serine) and further peptide structures such as palmitoylated pentapeptides (e.g. Matrixyl from Sederma) or the oligopeptide with the trade name Vincipeptide (from Vincience/France), and substances such as asiatic acid, madecassic acid, madecassoside, asiaticoside, extracts of Aloe and Centella species, niacinamide, astaxanthine, glucans e.g. from yeasts and oats, soy extracts and soy isoflavones, such as genistein and daidzein and furthermore rutin, chrysin, morin, betel nut alkaloids, forskolin, betulinic acid, extracts of Plantago species, TGF-beta, extracts of Ginkgo biloba, glutamine and glycolic acid. In most cases, however, only little is known about the biological mechanism of the collagen synthesis boosting effect.

The use of ternary mixtures containing Aloe extract, at least one substance selected from the group ascorbic acid and the derivatives thereof and at least one rosaceous plant extract, preferably raspberry leaf extract, strawberry leaf extract, blackberry leaf extract or meadowsweet herb extract for boosting collagen synthesis has, in contrast, not to date been reported in the literature.

Our own investigations have surprisingly shown that in particular mixtures containing Aloe extract, at least one substance selected from the group ascorbic acid and the derivatives thereof and at least one rosaceous plant extract, preferably raspberry leaf extract, strawberry leaf extract, blackberry leaf extract or meadowsweet herb extract have excellent collagen synthesis boosting characteristics. Activity was here synergistically enhanced in comparison with products consisting solely of Aloe extract, solely of at least one substance selected from the group ascorbic acid and the derivatives thereof or solely of at least one rosaceous plant extract, preferably raspberry leaf extract, strawberry leaf extract, blackberry leaf extract or meadowsweet herb extract. It proved possible unambiguously to demonstrate the synergistically enhanced activity of the ternary mixtures by calculating synergy index (SI) values with the assistance of Kull's equation (literature: F. C. Kull et al, Applied Microbiology Vol. 9, p. 538-541 (1961); D. C. Steinberg, Cosmetics & Toiletries Vol. 115(11), 59-62 (2000)).

A further, unexpected advantage of the mixtures according to the invention is their slight intrinsic odor, as a result of which they may particularly advantageously be used in cosmetics and especially in leave-on products, since the mixtures according to the invention accordingly bring about scarcely any or absolutely no changes to the appearance and odor of a cosmetic preparation containing even high concentrations of the extract. The mixtures additionally have weak, pleasant intrinsic taste. This additionally makes the mixtures suitable for use in oral hygiene products, since, in the case of use in the oral cavity, taste is an important acceptance criteria for users and is thus ultimately decisive with regard to the success of using the oral hygiene product.

The extract of Aloe plant material is particularly preferably produced from Aloe barbadensis and Aloe vera.

Preferred ascorbic acid derivatives are: sodium ascorbyl phosphate, magnesium ascorbyl phosphate, 3-O-ethyl ascorbic acid, allantoin ascorbate, aminopropyl ascorbyl phosphate, ascorbyl palmitate, araboascorbic acid monosodium salt, ascorbic acid polypeptide, ascorbosilane C, ascorbyl dipalmitate, ascorbyl alpha-glucoside, ascorbyl beta-glucoside, ascorbyl inositol nicotinate, ascorbyl linoleate, ascorbyl methylsilanol pectinate, ascorbyl nicotinamide, ascorbyl stearate, ascorbyl tetraisopalmitate, ascorbyl tocopherol maleate, calcium ascorbate, chitosan ascorbate, D-arabino-ascorbic acid, disodium ascorbyl sulfate, glucosamine ascorbate, inositol hexanicotinate hexaascorbate, isoascorbic acid, L-ascorbic acid, 2-(dihydrogen phosphate), 2-[3-cholest-5-en-3-yl hydrogen phosphate], 2-O-D-glucopyranosyl-, L-ascorbic acid, 3-O-ethyl ether, magnesium ascorbate, magnesium ascorbylborate, methoxy PEG-7 ascorbic acid, methylsilanol ascorbate, potassium ascorbyl tocopheryl phosphate, potassium ascorbylborate, sodium ascorbate, sodium ascorbyl/cholesteryl phosphate, sodium isoascorbate, sodium L-ascorbyl-2-phosphate, tetrahexyldecyl ascorbate, rosaceous plant extracts are here preferably obtained with a extracting agent consisting of pure water, pure ethanol or of ethanol/water mixtures in any desired quantity ratios from 99:1 (99 parts by weight of ethanol mixed with 1 part by weight of water) to 1:99 (1 part by weight of ethanol mixed with 99 parts by weight of water). The extracts are preferably further processed into pulverulent products by conventional, gentle drying processes such as e.g. spray drying or vacuum belt drying. Carriers such as e.g. maltodextrin may here be added in the concentration range from 10% to 95% and preferably in the concentration range from 30% to 80%.

All the stated active ingredients may be produced in either crystalline or powder form. Preferred methods for producing the ternary mixtures according to the invention are therefore any methods usual in the cosmetics industry and the pharmaceuticals industry for producing stable powder mixtures having a homogeneous distribution. It may, however, furthermore also be advantageous to produce the ternary mixtures according to the invention in the form of solutions. Particularly suitable solvents are here water, monohydric alcohols comprising from 1 to 5 C atoms such as in particular ethanol, propanol and butanol and polyhydroxylated aliphatic compounds such as in particular glycerin, propylene glycol, butylene glycol and 1,2-alkanediols having from 3-10 C atoms.

The concentration ratios of the ternary mixtures according to the invention containing in each case at least one of components a) to c) may here be selected as desired in the following manner, the following quantity distributions being preferred according to the invention:

component a): 0.01 weight percent to 99 weight percent and/or
component b): 0.01 weight percent to 99 weight percent and/or
component c): 0.01 weight percent to 99 weight percent, in each case relative to the total of components a), b) and c).

Preferred synergistically active ternary mixtures, on the other hand, contain an excess of component a), a moderate amount of component b) and, relative to components a) and b), a small amount of component c). Preferred mixtures are therefore those in which the proportions amount to,

component a): 33-99 wt. %
component b): 0.99-32.99 wt. %
component c): 0.01-10 wt. %, in each case relative to the total of components a), b) and c).

Synergistically boosted collagen synthesis may in particular be brought about with these quantity ratios.

Particularly preferred synergistically active ternary mixtures contain components a) to c) in the following quantity distribution:

component a): 80 to 98 wt. %
component b): 1.9 to 15 wt. %
component c): 0.1 to 4 wt. %

A very particularly preferred synergistically active ternary mixture contains components a) to c) in the following quantity distribution:

component a): 96.51 wt. %
component b): 3.22 wt. %
component c): 0.27 wt. %, in each case relative to the total of components a), b) and c).

The following composition is particularly preferred as a synergistically active ternary mixture containing raspberry leaf extract:

component a): 85-92 wt. %, preferably 88-92 wt. %, particularly preferably 90 wt. %,
component b): 7-10 wt. %, preferably 9 wt. %,
component c): 0.5-2 wt. %, preferably 0.8-1.2 wt. %, particularly preferably 1 wt. %, in each case relative to the total of components a), b) and c), with component b) consisting of magnesium ascorbyl phosphate and component c) of raspberry leaf extract.

A synergistically active ternary mixture which is particularly preferred in this connection contains components a) to c) in the following quantity distribution:

component a): 90.00 wt. %
component b), namely magnesium ascorbyl phosphate: 9.00 wt. %
component c), namely raspberry leaf extract: 1.00 wt. %, in each case relative to the total of components a), b) and c).

The concentration of the ternary mixtures (in liquid and/or concentrated form) in cosmetic, oral hygiene and/or pharmaceutical preparations (in particular for topical application) is preferably in the range from 0.001 to 20 wt. %, preferably in the range from 0.01 to 10 wt. % and particularly preferably in the range from 0.1 to 5 wt. %, relative to the entire preparation. These concentrations are particularly preferred for synergistically stimulating collagen synthesis. They are furthermore preferred
for slowing down skin ageing and/or
for preventing and/or slowing down periodontitis and/or caries.

The mixtures according to the invention are preferably produced according to the invention by a method involving mixing components a), b) and c) in their respectively selected amounts to obtain a homogeneous pulverulent mixture.

The ternary mixtures according to the invention in powder or liquid form may preferably be further processed to yield a final product, likewise according to the invention, in preferably liquid form, by adding to the production method according to the invention the step: combining the ternary mixture with a further solvent or carrier which is acceptable for pharmaceutical, oral hygiene and/or cosmetic purposes.

The collagen synthesis boosting ternary mixtures according to the invention are surprisingly ideally suitable as cosmetic and/or pharmaceutical agents for preventing, alleviating and treating unwanted changes in the oral cavity which are attributable to elevated activity of MMPs and the concomitant degradation of collagen, since they compensate the negative effects of the MMPs by boosting de novo synthesis of collagen. Combined use of the collagen synthesis boosting mixtures according to the invention together MMP inhibitors which are not according to the invention may here likewise be advantageous.

For the purposes of the present invention, a carrier which is acceptable for pharmaceutical, oral hygiene or cosmetic purposes is a carrier which is at least nontoxic for the organism on which it is to be used. Preferred solids which are suitable for cosmetic, oral hygiene or pharmaceutical purposes are pulverulent maltodextrin, lactose, silicon dioxide or glucose and mixtures of two or more of these solids. An acceptable solid which is particularly preferred according to the invention for oral hygiene purposes is silicon dioxide. Further acceptable solids which may be used according to the invention for oral hygiene purposes are hydrocolloids such as starches, degraded starches, chemically or physically modified starches, modified celluloses, gum arabic, gum ghatti, tragacanth, karaya, carrageenan, pullulan, curdlan, xanthan gum, gellan gum, guar flour, locust bean flour, alginates, agar, pectin and inulin together with mixtures of two or more of these solids, in particular also with silicon dioxide.

The solid or liquid ternary mixture may also be further processed according to the invention to yield a preferably liquid preparation by [mixing] the ternary mixture with a further solvent selected from the group consisting of glycerin, 1,2-propylene glycol, 1,3-butylene glycol, ethanol, water and mixtures of two or more of the stated solvents with water. Such final products produced according to the invention may in particular readily be further processed for cosmetic and oral hygiene purposes. These preparations according to the invention may optionally be produced with addition of a preservative, solubilizing agent or antioxidant. Furthermore, the solid or liquid ternary mixtures according to the invention or the liquid or solid preparation also according to the invention containing a solid or liquid ternary mixture according to the invention may be further processed by encapsulation. According to the invention, the solid or liquid ternary mixtures according to the invention or the liquid or solid preparation containing a solid or liquid ternary mixture according to the invention are encapsulated with a solid shell material which is preferably selected from starches, degraded or chemically or physically modified starches (in particular dextrins and maltodextrins), gelatins, gum arabic, agar-agar, gum ghatti, gellan gum, modified and unmodified celluloses, pullulan, curdlan, carrageenans, alginic acid, alginates, pectin, inulin, xanthan gum and mixtures of two or more of the stated substances.

The solid shell material is preferably selected from gelatin (pig, cattle, chicken and/or fish gelatins and mixtures thereof are advantageous, preferably comprising at least one gelatin with a Bloom value of greater than or equal to 200, preferably with a Bloom value of greater than or equal to 240), maltodextrin (preferably obtained from maize, wheat, tapioca or potato, preferred maltodextrins have a DE value in the range from 10-20), modified cellulose (e.g. cellulose ethers), alginates (e.g. Na alginate), carrageenan (beta-, iota-, lambda- and/or kappa-carrageenan), gum arabic, curdlan and/or agar-agar. Gelatin is in particular preferably used thanks to its readily availability in different Bloom values. In particular, seamless gelatin or alginate capsules, the shell of which dissolves very quickly in the mouth or bursts when chewed and so releases the active ingredient into the oral cavity are particularly preferably used for oral hygiene purposes. Production may proceed, for example, as described in EP 0 389 700, JP 7 196 478, U.S. Pat. No. 4,251,195, U.S. Pat. No. 6,214,376, WO 03/055587 or WO 2004/050069.

The synergistically active ternary mixture containing at least one extract of an Aloe species selected from a), at least one substance from the group of ascorbic acid and the derivatives thereof selected from b) and at least one rosaceous plant extract, preferably raspberry leaf extract, strawberry leaf extract, blackberry leaf extract or meadowsweet herb extract selected from c) may advantageously be used in cosmetics in any applications in which a boost in collagen synthesis is associated with cosmetically desired effects. Preferably, however, the mixture is used as an active ingredient against natural and premature, for example sunlight-induced, skin ageing and against wrinkles. To this end, it is preferably applied topically onto the skin to be treated.

Substantial areas of application are here cosmetic, in particular dermatological preparations, which (apart from components a)-c) according to the invention) are of conventional composition and cosmetic, in particular dermatological photoprotection, for the treatment, care and cleaning of skin and/or hair or as a makeup product in decorative cosmetics. Depending on their composition, such preparations may accordingly for example be used as a skin protection cream, day or night cream, eye cream, sunscreen lotion or aftersun lotion, nourishing cream, care mask, gel pads, face lotion, moist care and cleansing wipes, cleansing milk, cleansing soap, foam bath or shower gel, deodorant, antiperspirant, shampoo, hair care product, hair conditioner, hair color, hair styling agent and preferably take the form of an emulsion, lotion, milk, fluid, cream, hydrodispersion gel, balm, spray, alcoholic or aqueous/alcoholic solution, foam, powder, liquid soap, tablet soap, shampoo, roll-on, stick, or makeup. In hair treatment agents, use preferably focuses on the scalp.

It has moreover been found that a synergistically active ternary mixture according to the invention in a concentration sufficient to stimulate collagen synthesis has an action for treating periodontitis and caries and in particular prevents and delays the progress thereof. The invention accordingly teaches advantageously making use of synergistically active ternary mixtures according to the invention and corresponding oral care products in any oral care or oral hygiene applications in which a boost in collagen synthesis is associated with desired effects. Preferably, however, the synergistically active ternary mixture or the cosmetic, pharmaceutical or oral care preparation is used as an active ingredient for preventing, slowing down and healing periodontitis and caries, for treating infections of the oral cavity and for wound healing in the oral cavity. To this end, the ternary mixture according to the invention is preferably brought into contact externally with the oral mucosa and the teeth. Such mixtures and oral care formulations may in particular be used to this end according to the invention to clean the hard tooth structure and the oral cavity and to heal infective inflammation and wounds in the oral cavity.

The concentration of the ternary mixtures (in liquid and/or concentrated form) in cosmetic, oral hygiene and/or pharmaceutical preparations (in particular for topical application) is preferably in the range from 0.001 to 20 wt. %, preferably in the range from 0.01 to 10 wt. % and particularly preferably in the range from 0.1 to 5 wt. %, relative to the entire preparation. The mixtures according to the invention may straightforwardly be incorporated into conventional cosmetic or dermatological formulations such as pump sprays, aerosol sprays, creams, ointments, tinctures, lotions, nail care products and the like. It is here also possible and in many cases advantageous to combine a blackberry leaf extract used according to the invention with further active ingredients, for example with other active ingredients against skin ageing and wrinkles. These cosmetic and/or dermatological formulations containing blackberry leaf extract may here otherwise be of conventional composition and serve for treatment of the skin and/or the hair in the manner of dermatological treatment or treatment in the manner of cosmetic care preparations. They may, however, also be used in makeup products in decorative cosmetics.

Preparations according to the invention containing the ternary mixtures according to the invention may also contain further collagen synthesis boosting active ingredients and active ingredient combinations in particular against skin ageing and wrinkles or for oral care (in particular against periodontitis and caries, against inflammation of the oral mucosa and for wound healing. According to the invention, any active ingredients suitable or conventional for oral hygiene, for cosmetic and/or dermatological applications may be used. Particularly preferred ingredients are peptide substances and the derivatives thereof such as e.g. carnitine, carnosine, creatine, matrikine peptides (e.g. lysyl-threonyl-threonyl-lysyl-serine) and further peptide structures such as palmitoylated pentapeptides (e.g. Matrixyl from Sederma) or the oligopeptide with the trade name Vincipeptide (from Vincience/France), substances such as asiatic acid, madecassic acid, madecassoside, asiaticoside, extracts of Centella species, niacinamide, astaxanthine, glucans e.g. from yeasts and oats, soy extract and soy isoflavones, such as genistein and daidzein, furthermore rutin, chrysin, morin, betel nut alkaloids, forskolin, betulinic acid, extracts of Plantago species, extracts of Ginkgo biloba, catechin (catechin (e.g. epigallocatechin gallate)-rich extracts of green or black tea), polyphenois (oligomeric procyanidins) from wine, retinoic acid, retinal, retinol, retinol palmitate, and further retinol derivatives, glutamine, glycolic acid and TGF-beta.

In oral hygiene applications, the ternary mixtures according to the invention may be incorporated into the most varied dosage forms without being limited to one or a few specific dosage forms, as they advantageously combine harmoniously with a large number of conventional auxiliary substances and additives. It is here advantageous to buffer the oral hygiene products according to the invention. A pH range from 3.5 to 10.0 is particularly advantageous and preferred. Preferred oral hygiene products are preferably tooth creams, toothpastes, tooth gels, mouthwashes, mouth rinses, liquids for gargling and mouth or throat sprays and suckable pastilles, suckable tablets, candies, chewing gums, chewable candies and chewing gums for dental care.

It is also possible and usually advantageous for oral hygiene purposes to combine a ternary mixture according to the invention with other materials, in particular with MMP inhibitors, but also for example with antimicrobially active or antiinflammatory substances, aroma substances, flavorings and/or auxiliary substances.

MMP inhibitors which may preferably be used in combination are retinyl palmitate, propyl gallate, precocene, 6-hydroxy-7-methoxy-2,2-dimethyl-1(2H)-benzopyran, 3,4-dihydro-6-hydroxy-7-methoxy-2,2-dimethyl-1 (2H)-benzopyran, benzamidine hydrochloride, the cysteine proteinase inhibitors N-ethylmaleimide and epsilon-amino-n-caproic acid, the serine protease inhibitor: phenylmethylsulfonyl fluoride, Collhibin (from Pentapharm; INCI: hydrolyzed rice protein) Oenotherol (from Soliance; INCI; propylene glycol, aqua, Oenothera biennis root extract, ellagic acid, and ellagitannins, (e.g. from pomegranate), phosphoramidon hinokitiol, EDTA, galardin, EquiStat (from Collaborative Group; apple fruit extract, soy seed extract: ursolic acid, soy isoflavones & soy proteins), sage extracts, MDI (from Atrium; INCI: glycosaminoglycans), Fermiskin (from Silab/Mawi; INCI: water and Lentinus edodes extract), Actimp 1.9.3. (from Expanscience/Rahn; INCI: hydrolyzed lupine protein), Lipobelle soy glycone (from Mibelle; INCI: alcohol, polysorbate 80, lecithin and soy isoflavones), extracts of green and black tea and numerous further plant extracts which are listed in WO 02/069992 (see tables 1-12).

The oral hygiene products according to the invention may furthermore contain auxiliary materials as are conventionally used in such preparations, e.g. preservatives, abrasives, antibacterial agents, antiinflammatory agents, irritation-preventing agents, irritation-suppressing agents, antimicrobial agents, antioxidants, astringents, antiseptic agents, antistatic agents, binders, buffers, excipients, chelating agents, cell stimulants, cleaning agents, care agents, surface-active substances, deodorizing agents, plasticizers, bactericides, emulsifiers, enzymes, essential oils, film formers, fixatives, foaming agents, foam stabilizers, substances for preventing foaming, foam boosters, gelling agent, gel-forming agents, moisture-donating agents, moisturizing substances, moisture-retaining substances, bleaching agents, optical brighteners, soil-repelling agents, friction-reducing agents, slip agents, opacifiers, hiding agents, lustrants, polymers, powders, proteins, abrasive agents, silicones, skin-soothing agents, skin-cleaning agents, skin-conditioning agents, skin-healing agents, cooling agents, skin-cooling agents, warming agents, skin-warming agents, stabilizers, suspending agents, thickeners, vitamins, oils, waxes, fats, phospholipids, saturated fatty acids, mono- or polyunsaturated fatty acids, α-hydroxy acids, polyhydroxyfatty acids, liquefying agents, dyes, color-protective agents, pigments, aromas, flavorings, odoriferous substances or other conventional constituents of an oral hygiene formulation such as alcohols, polyols, electrolytes, organic solvents, sweeteners, sugar substitutes, silicas, calcium carbonate, calcium hydrogenphosphate, aluminum oxide, fluorides, zinc, tin, potassium, sodium and strontium salts, pyrophosphates, hydrogen peroxide, hydroxyapatites.

If the oral hygiene product is a solution or lotion, the solvents stated in WO2005/123101 may, for example, be used. Mixtures of the above-stated solvents may, of course, also be used.

Examples of flavorings or aromas which, in addition to a ternary mixture according to the invention, may be a constituent of an oral hygiene product according to the invention are listed e.g. in K. Bauer, D. Garbe, H. Surburg, Common Fragrance and Flavor Materials, 4th. Ed., Wiley-VCH, Weinheim 2001 or also in S. Arctander, Perfume and Flavor Chemicals, Vol. I and II, Montclair, N.J., 1969, private publication.

Examples of natural aromas which, in addition to a ternary mixture according to the invention, may be a constituent of an oral hygiene product according to the invention are stated in WO2005/123101.

Examples of uniform aromas which, in addition to a ternary mixture according to the invention, may be a constituent of an oral hygiene product according to the invention are likewise stated in WO2005/123101.

Compounds with a physiological cooling effect (cooling substances) which, in addition to a ternary mixture according to the invention, may be a constituent of an oral hygiene product according to the invention and/or of a cosmetic, dermatological and/or pharmaceutical product according to the invention, are stated in WO2005/123101. Oral hygiene products which are preferred according to the invention are those which, in addition to l-menthol, contain at least one, particularly preferably at least two further cooling substances.

Components which cause a warming, spicy, tingling or prickling sensation on the skin or mucous membranes, in particular aroma substances with a warming effect and/or pungent tasting compounds (pungent substances) and which, in addition to a ternary mixture according to the invention, may be a constituent of an oral hygiene product and/or cosmetic, dermatological or pharmaceutical product according to the invention are likewise stated in WO2005/123101.

Further components which, in addition to a ternary mixture according to the invention, may be a constituent of an oral hygiene product according to the invention are e.g. substances for improving oral hygiene, such as for example dental care and/or refreshing substances. Substances for improving oral hygiene include, for example, substances for combating or preventing plaque, calculus or caries and those for combating or preventing bad breath. Reference is made in this connection to U.S. Pat. No. 5,043,154. Examples of substances which are to be used are furthermore stated in WO2005/123101.

An oral hygiene product according to the invention may, in addition to a ternary mixture according to the invention, also contain one or more antimicrobial active ingredients for improving oral hygiene. These active ingredients may be of a hydrophilic, amphoteric or hydrophobic nature. Examples of such active ingredients are likewise stated in WO2005/123101.

A cosmetic, dermatological or pharmaceutical. product and/or oral hygiene product according to the invention may, in addition to a ternary mixture according to the invention, also contain dyes, colorants or pigments which are likewise stated in WO2005/123101.

Preparations serving for cosmetic, for oral hygiene and/or for pharmaceutical purposes which contain a ternary mixture according to the invention may particularly advantageously contain antioxidants, it being possible to use any antioxidants which are suitable for or usual in oral hygiene, cosmetic and/or dermatological applications. The antioxidants selected are advantageously those stated in WO2005/123101.

Preferred preparations serving for oral hygiene, cosmetic and/or pharmaceutical purposes which contain a ternary mixture according to the invention also contain one or more vitamins and/or vitamin precursors, it being possible to use any vitamins and vitamin precursors which are suitable for or usual in cosmetic and/or dermatological applications. These include in particular the vitamins and vitamin precursors already stated in WO2005/123101.

For use in the manner conventional for cosmetics and skin preparations, the ternary mixtures according to the invention are applied in an adequate amount onto the skin and/or the hair. Cosmetic and dermatological preparations which contain a ternary mixture according to the invention and additionally act as a sunscreen preparation offer particular advantages. These preparations advantageously contain at least one UVA filter and/or at least one UVB filter and/or at least one inorganic pigment. The preparations may here assume various forms, as they are e.g. conventionally used for sunscreen preparations. They may accordingly form e.g. a solution, an emulsion of the water-in-oil (W/O) or oil-in-water (O/W) type, or a multiple emulsion, for example of the water-in-oil-in-water (W/O/W) type, a gel, a hydrodispersion, a solid stick or also an aerosol.

Preparations according to the invention in the field of cosmetics and skin preparations which contain a ternary mixture according to the invention are particularly advantageously combined with substances which absorb or reflect UV radiation, specifically for cosmetic or skin-protecting purposes (thus not for oral hygiene purposes), the total amount of the filter substances amounting to from 0.01 wt. % to 40 wt. %, preferably 0.1% to 10 wt. %, in particular 1.0 to 5.0 wt. %, relative to the total weight of the preparations, in order to provide cosmetic preparations which protect the hair or skin from ultraviolet radiation. These preparations advantageously contain at least one UVA filter and/or at least one UVB filter and/or at least one inorganic pigment, such that a sun protection factor of at least >2 (preferably >5) is achieved. These preparations according to the invention may here assume various forms, as are e.g. conventionally used for sunscreen preparations. They may accordingly be e.g. a solution, an emulsion of the water-in-oil (W/O) or oil-in-water (O/N) type, or a multiple emulsion, for example of the water-in-oil-in-water (W/O/W) type, a gel, a hydrodispersion, a solid stick or also an aerosol.

Advantageous UV filters and inorganic photoprotective pigments are stated in WO2005/123101. UV absorbers which are particularly suitable for combining are likewise stated in WO2005/123101.

Advantageous inorganic photoprotective pigments are finely dispersed metal oxides and metal salts which are likewise stated in WO2005/123101. The total amount of inorganic pigments, in particular hydrophobic inorganic micropigments in the finished cosmetic or dermatological formulations is advantageously in the range from 0.1 to 30 wt. %, preferably 0.1 to 10.0, in particular 0.5 to 6.0 wt. %, relative to the total weight of the formulations.

In many preparations, a combination with (metal) chelating agents may also be advantageous. (Metal) chelating agents which are preferably to be used are those compounds stated in WO2005/123101.

Cosmetic, pharmaceutical and/or oral hygiene preparations which are preferred according to the invention may also contain antiinflammatory active ingredients and/or active ingredients which relieve erythema and/or itching. Any antiinflammatory active ingredients and any active ingredients which relieve erythema and/or itching which are suitable for cosmetic and/or dermatological and/or oral hygiene applications may here be used. The compounds stated in WO2005/123101 are advantageously used as antiinflammatory active ingredients or active ingredients which relieve erythema and/or itching.

The amount of antirritants (one or more compounds) in the preparations according to the invention preferably amounts to 0.0001 to 20 wt. %, particularly preferably 0.0001-10 wt. %, in particular 0.001-5 wt. %, relative to the total weight of the preparation.

Ternary mixtures according to the invention may likewise advantageously be used for cosmetic and/or dermatological purposes in combination with skin-lightening active ingredients. Any skin-lightening active ingredients suitable for or usual in cosmetic and/or dermatological applications which are stated in WO2005/123101 may here be used according to the invention.

Ternary mixtures according to the invention may in particular advantageously be used in cosmetic and dermatological preparations in combination with insect repellents, such as e.g. DEET, IR 3225, Dragorepel (Symrise GmbH & Co. KG).

Ternary mixtures according to the invention may furthermore advantageously be used, in particular in cosmetic and dermatological preparations, in combination with hair growth inhibitors as stated in WO2005/123101.

In numerous cases, ternary mixtures according to the invention may also advantageously be used in combination with the osmolytes stated in WO2005/123101.

Ternary mixtures according to the invention may advantageously be used, in particular in cosmetic and dermatological preparations, in combination with hair care products and antidandruff active ingredients (e.g. climbazole, ketoconazole, piroctone olamine, zinc pyrithione).

Ternary mixtures according to the invention may also in numerous cases advantageously be used in preparations according to the invention in combination with one or more preservatives. The preservatives selected are here preferably those stated in WO2005/123101.

It is additionally preferred according to the invention to use ternary mixtures according to the invention in combination with substances which are primarily used for inhibiting the growth of undesired microorganisms on or in animal organisms. Apart from conventional preservatives, further active ingredients which may be mentioned in addition to the large group of conventional antibiotics are in particular the compounds stated in WO2005/123101.

Ternary mixtures according to the invention may furthermore also particularly advantageously be used according to the invention in combination with antiperspirant active ingredients (antiperspirants) for combating body odor in cosmetic, dermatological and/or pharmaceutical preparations. Antiperspirant active ingredients which may primarily be considered are the compounds stated in WO2005/123101.

Ternary mixtures according to the invention may advantageously be combined in cosmetic and dermatological preparations with cosmetic auxiliary substances as are conventionally used in such preparations, thus e.g. with: perfume oils; agents for preventing foaming; dyes; pigments having a coloring effect; thickeners; surface-active substances; emulsifiers; softening substances; moistening and/or moisture-retaining substances; fats; oils; waxes; other conventional constituents of a cosmetic formulation such as alcohols, polyols, polymers, foam stabilizers, electrolytes, organic solvents or silicone derivatives.

A large proportion of care substances is often advantageous in formulations for topical prophylactic or cosmetic treatment of the skin which contain ternary mixtures according to the invention. According to a preferred embodiment, the compositions contain one or more of the stated animal- and/or plant-based care products stated in WO2005/123101.

The animal and/or plant protein hydrolysates stated in WO2005/123101 may advantageously also be added to the ternary mixture according to the invention.

Where a cosmetic or dermatological preparation containing a ternary mixture according to the invention is a solution or lotion, the solvents stated in WO2005/123101 may advantageously be used.

Cosmetic preparations according to the invention which contain a ternary mixture according to the invention may advantageously also contain the moisture-retaining regulators stated in WO2005/123101.

In preferred embodiments of the invention, cosmetic preparations which contain a ternary mixture according to the invention may also contain mono-, di- and oligosaccharides.

It is furthermore preferred for cosmetic preparations which contain a ternary mixture according to the invention to contain one or more further plant extracts which are conventionally produced by extracting the entire plant, but in individual cases also just flowers and/or leaves, wood, bark or roots of the plant. With regard to the plant extracts usable for cosmetic, pharmaceutical and/or dermatological purposes, a person skilled in the art will refer to the table beginning on page 44 of the 3rd edition of the “Leitfaden zur Inhaltsstoffdeklaration kosmetischer Mittel” [“Guidelines for the nomenclature of ingredients in cosmetic agents”], published by the German Cosmetic, Toiletry, Perfumery and Detergent Association (IKW), Frankfurt. The extracts stated in WO2005/123101 are advantageously suitable for cosmetic, dermatological, pharmaceutical and/or oral hygiene purposes.

According to the invention, cosmetic preparations which contain a ternary mixture according to the invention may, in particular if crystalline or microcrystalline solids, for example inorganic micropigments, are to be incorporated into the preparations, may also contain the anionic, cationic, nonionic and/or amphoteric surfactants stated in WO2005/123101.

The surface-active substance may be present in the preparations according to the invention in a concentration of between 1 and 98 wt. %, relative to the total weight of the preparations.

Cosmetic or dermatological preparations which contain a ternary mixture according to the invention may also assume emulsion form.

The oil phase of cosmetic, dermatological and/or pharmaceutical preparations according to the invention may advantageously be selected from groups of substances stated in WO2005/123101.

Preparations according to the invention which assume emulsion form advantageously comprise one or more emulsifiers. O/W emulsifiers may, for example, advantageously be selected from the group of products stated in WO2005/123101.

Preferred developments and further aspects of the present invention are revealed by the attached claims and the following Examples together with the tables, the examples not being intended to limit the invention:

### EXAMPLE 1

Method for determining the collagen synthesis boosting activity of selected substances of natural or synthetic origin.

Fibroblasts are responsible for collagen synthesis in the dermis layer of the skin. The test system involves culturing normal human dermal fibroblasts (NHDF) in a “monolayer” culture in 96 well MTPs, incubating them over a defined period with test substances and then determining the increase in collagen qualitatively by a staining method.

The principle of the assay is based on histological, specific staining of the soluble collagen (predominantly type I and III collagen) with the assistance of the dye Sirius Red (Direct Red 80). Sirius Red is an anionic dye having sulfonic acid groups attached to its side chains. These sulfonic acid groups react with the side chain groups of the amino acids (proline, hydroxyproline, lysine and hydroxylysine) predominantly occurring in collagen, it being necessary for an intact collagen structure to be present.

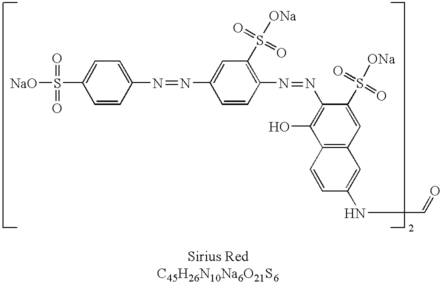

The model used for the assay design is the procedure described in B. J. Walsh et al., Analytical Biochemistry 203, 187-190 (1992) for the quantitative determination of collagen on the 96 well scale.

The test substance's collagen synthesis boosting effect is tested on in vitro aged (>8th passage) NHDF in 96 well microtiter plates (2*10⁴ cells per well). A suitable solvent must first be identified for each test sample by preparing a stock solution of the test sample. The incubation solutions of the test substance are then produced in DMEM supplemented with 0.2% BSA. Possible solvents should be present in an amount of at most 0.2% in all dilutions of the incubation solution. The maximum usage concentration of the test substance is based on the IC₂₀ values obtained in the cytotoxicity investigation (resazurin assay on 3T3 mouse fibroblasts) and is set at 0.1*IC₂₀.

After a 24 h adaptation phase for the NHDF, the cells are incubated (37° C., 5% CO₂) for 48 h with the incubation solution and corresponding control solutions (solvent and blank). In order to be able to identify possible influences of the test substances on the subsequent staining procedure, the highest usage concentration of the test substances is also simultaneously processed without cells as a background control. The cells are then disrupted by a double freeze/thaw cycle (−20° C./−80° C./37° C./−80° C./37° C.; 10 minutes each) and 96 hours' evaporation (24 h moist heat at 37° C., 72 h dry heat at 30° C.). The resultant collagen-coated wells can be stained the Sirius Red dye (0.1% solution). The dye bound to the collagen is brought back into solution by a triple washing step with 0.01 mM HCl solution and subsequent incubation (at least 5 minutes) with 0.1 mM NaOH solution. The absorption of the resultant dye solution can be determined photometrically at 540 nm.

Thanks to the linear relationship between collagen content and the OD values arising from staining, the increase in OD is determined for qualitative assessment of the collagen synthesis boosting effect. To this end, the mean and standard deviations of the controls and the test samples are calculated and then a background or blank correction is made. The increase in OD is then obtained from the relationship between the cells which have been treated (with test substance) and those which have not been treated (only with solvent or medium).

### Sources:

1. Walsh B. J., Thornton S. C., Penny R. and Breit S. N.: Microplate Reader-Based Quantitation of Collagens; Analytical Biochemistry 203, 187-190 (1992)
2. Nethery A., Lyons J. G. and O'Grady R. L.: A Spectrometric Collagenase Assay: Analytical Biochemistry 159, 390-395 (1986)

### EXAMPLE 2

Determination of the synergistically enhanced collagen synthesis boosting activity of selected ternary mixtures containing at least one extract of an Aloe species, at least one substance from the group ascorbic acid and the derivatives thereof and at least one rosaceous plant extract, preferably raspberry leaf extract, strawberry leaf extract, blackberry leaf extract or meadowsweet herb extract.

The potentially synergistically enhanced activities of ternary mixtures containing Aloe extract, at least one substance from the group ascorbic acid and the derivatives thereof and at least one rosaceous plant extract, preferably raspberry leaf extract, strawberry leaf extract, blackberry leaf extract or meadowsweet herb extract were determined by ascertaining the collagen synthesis boosting characteristics both for the individual substances and for different ternary mixtures in accordance with the test method described in Example 1. The results of the investigations are listed in Table 1.

TABLE 1
Collagen synthesis boost of magnesium ascorbyl phosphate, Aloe
extract, raspberry leaf extract, strawberry leaf extract, meadowsweet herb
extract and ternary mixtures containing (i) magnesium ascorbyl phosphate,
Aloe extract and meadowsweet herb extract, (ii) magnesium ascorbyl
phosphate, Aloe extract and strawberry leaf extract, (iii) magnesium
ascorbyl phosphate, Aloe extract and raspberry leaf extract and (iiii)
magnesium ascorbyl phosphate, Aloe extract and blackberry leaf extract;
boost in % relative to untreated control

| | | Collagen | |
|---|---|---|---|
| | | synthesis boost | |
| | Usage concentration | [%], relative to | Synergy |
| | [mg/ml] | untreated control | index SI |
| Aloe extract (Bio505) | 10.860 | 45.70 | —/— |
| Magnesium ascorbyl | 0.362 | 66.97 | —/— |
| phosphate (Bio172) | | | |
| Meadowsweet herb | 0.030 | 14.22 | —/— |
| extract (Bio1544) | | | |
| Ternary mixture | 3.620 (Bio505) + | 112.76 | 3.99 |
| (Bio505 + Bio172 + | 0.121 (Bio172) + | | |
| Bio1544) | 0.010 (Bio1544) | | |
| Aloe extract (Bio505) | 10.860 | 45.70 | —/— |
| Magnesium ascorbyl | 0.362 | 66.97 | —/— |
| phosphate (Bio172) | | | |
| Strawberry leaf | 0.030 | 14.85 | —/— |
| extract (Bio1545) | | | |
| Ternary mixture | 3.620 (Bio505) + | 96.37 | 3.31 |
| (Bio505 + Bio172 + | 0.121 (Bio172) + | | |
| Bio1545) | 0.010 (Bio1545) | | |
| Aloe extract (Bio505) | 10.860 | 47.59 | —/— |
| Magnesium ascorbyl | 0.362 | 67.44 | —/— |
| phosphate (Bio172) | | | |
| Raspberry leaf | 0.030 | 16.74 | —/— |
| extract (Bio 615) | | | |
| Ternary mixture | 3.620 (Bio505) + | 142.15 | 4.48 |
| (Bio505 + Bio172 + | 0.121 (Bio172) + | | |
| Bio615) | 0.010 (Bio615) | | |
| Magnesium ascorbyl | 0.362 | 66.97 | —/— |
| phosphate (Bio172) | | | |
| Aloe extract (Bio505) | 10.860 | 45.70 | —/— |
| Blackberry leaf | 0.003 | 14.15 | —/— |
| extract (Bio684/4) | | | |
| Ternary mixture | 3.620 (Bio505) + | 153.93 | 5.46 |
| (Bio505 + Bio172 + | 0.121 (Bio172) + | | |
| Bio684/4) | 0.010 (Bio684/4) | | |

### Result:

The synergy index (SI) values of the ternary mixtures according to the invention were calculated according to Kull (literature: F. C. Kull et al, Applied Microbiology vol. 9, p. 538-541 (1961); D. C. Steinberg, Cosmetics & Toiletries vol. 115(11), 59-62 (2000) by means of the following equation.

SI=Z*D/A+Z*E/B+Z*F/C

where:
D, E, F: proportion factor for the individual constituents (e.g., 33 weight percent=factor 0.33)
A, B, C: collagen stimulation of the individual substances (increase in %, relative to control without active ingredient)
Z: collagen stimulation of the ternary mixture (increase in %, relative to control without active ingredient)

A synergistic effect is present if values of greater than 1.00 are obtained. Values of less than 1.00 indicate an antagonistic effect. A value of exactly 1.00 indicates an effect which is neither synergistic nor antagonistic.

An example calculation is show below for the ternary mixture containing in each case one third (D, E and F factor in each case 0.33) of the amounts of magnesium ascorbyl phosphate, Aloe extract and raspberry leaf extract used in the individual experiment.

SI=142.15*033/67.44+142.15*0.33/47.59+142.15*0.33/16.74=4.48

The SI value of 4.48 shows a significant, synergistically enhanced effect of the mixture containing magnesium ascorbyl phosphate, Aloe extract and raspberry leaf extract. The SI values of the further investigated ternary mixtures are shown in Table 1.

The results shown in Table 1 and, in particular, the calculated synergy index values unambiguously show that the ternary mixtures according to the invention containing: Aloe extract, magnesium ascorbyl phosphate, and a rosaceous plant extract (according to Table 1 e.g. raspberry leaf extract, strawberry leaf extract, blackberry leaf extract or meadowsweet herb extract) at a quantity ratio of 96.51 weight percent Aloe to 3.22 weight percent magnesium ascorbyl phosphate, to 0.27% weight percent rosaceous plant extracts, exhibit a synergistically enhanced collagen synthesis boosting activity. In the case of the ternary mixture containing Aloe extract, magnesium ascorbyl phosphate and strawberry leaf extract, it proved possible to boost collagen synthesis by 96.37% when using in each case one third of the total amount (synergy index: 3.31). In the case of the ternary mixture containing Aloe extract, magnesium ascorbyl phosphate and meadowsweet herb extract, it proved possible to boost collagen synthesis by 112.76% when using in each case one third of the total amount (synergy index: 3.99). It proved possible to achieve the greatest boost in collagen synthesis by 142.15% or 153.93% when using in each case one third of the total amount for the ternary mixture containing Aloe extract, magnesium ascorbyl phosphate and raspberry leaf extract or blackberry leaf extract respectively (synergy index values: 4.48 and 5.46). Since synergy index values of greater than 1 were determined in every case, the synergistic effect of the ternary mixtures according to the invention has been unambiguously proven.

### EXAMPLE 3

Determination of the synergistically enhanced collagen synthesis boosting activity of further ternary mixtures containing (i) Aloe extract, (ii) magnesium ascorbyl phosphate or sodium ascorbyl phosphate and (iii) raspberry leaf extract:

Example 3 shows further ternary mixtures having mixture ratios modified relative to Example 2, in which it likewise proved possible to identify a synergistically enhanced activity of the ternary mixtures. The results of the investigations are listed in Table 2.

TABLE 2
Collagen synthesis boost of Aloe extract, magnesium ascorbyl
phosphate, sodium ascorbyl phosphate, raspberry leaf extract
and ternary mixture containing (i) Aloe extract, (ii) magnesium
ascorbyl phosphate or sodium ascorbyl phosphate and (iii)
raspberry leaf extract; boost in % relative to untreated control.

| | | Collagen | |
|---|---|---|---|
| | | synthesis boost | |
| | Usage concentration | [%], relative to | Synergy |
| | [mg/ml] | untreated control | index SI |
| Aloe extract (Bio505) | 10.110 | 39.87 | —/— |
| Magnesium ascorbyl | 1.020 | 141.13 | —/— |
| phosphate | | | |
| (Bio1300/1) | | | |
| Sodium ascorbyl | 1.020 | 144.57 | —/— |
| phosphate (Bio1586) | | | |
| Raspberry leaf | 0.12 | 9.93 | —/— |
| extract (Bio 615) | | | |
| Ternary mixture A | 3.370 (Bio505) + | 160.52 | 7.04 |
| (Bio505 + | 0.340 (Bio . . . ) + | | |
| Bio1300/1 + Bio615) | 0.040 (Bio615) | | |
| Ternary mixture B | 3.370 (Bio505) + | 94.95 | 4.16 |
| (Bio505 + Bio1586 + | 0.340 (Bio . . . ) + | | |
| Bio615) | 0.040(Bio615) | | |

### Result:

The results listed in Table 2 show that further ternary mixtures according to the invention containing: Aloe extract, magnesium ascorbyl phosphate, and raspberry leaf extract or Aloe extract, sodium ascorbyl phosphate and raspberry leaf extract in a quantity ratio of 90 weight percent Aloe extract to 9 weight percent magnesium ascorbyl phosphate (or 9 weight percent sodium ascorbyl phosphate) to 1 weight percent raspberry leaf extract likewise exhibit a synergistically enhanced, collagen synthesis boosting activity. In the case of the ternary mixture containing Aloe extract, magnesium ascorbyl phosphate and raspberry leaf extract, it proved possible to boost collagen synthesis by 160.52% (synergy index: 7.04) when using in each case one third of the tested amount of individual substances. In the case of the ternary mixture containing (ii) Aloe extract, sodium ascorbyl phosphate and raspberry leaf extract, it proved possible to boost collagen synthesis by 94.95% (synergy index 4.16) when using in each case one third of the tested amount of individual substances. Since synergy index values of greater than 1 were determined in both cases, the synergistic effect of the ternary mixtures according to the invention has been unambiguously proven.

### EXAMPLE 4

### Further Cosmetic/Dermatological Examples of Application

The dermatological and cosmetic preparations containing synergistically active ternary mixtures in combination with other cosmetic active ingredients and additives were used by being applied in a sufficient amount onto the skin and/or the hair in the manner conventional for cosmetics. Advantageous preparations for some applications are stated by way of example below.

### 4.1 Antiwrinkle W/O Cream

| | Raw material name | | Content |
|---|---|---|---|
| Part | (manufacturer) | INCI name | in wt. % |
| A | Dragosan W/O P | Sorbitan isostearate, | 8.00 |
| | | (Symrise) hydrogenated | |
| | | castor oil, ceresin, | |
| | | beeswax (cera alba) | |
| | Dragoxat EH (Symrise) | Ethylhexyl | 8.00 |
| | | ethylhexanoate | |
| | Isodragol (Symrise) | Triisononanoin | 8.00 |
| | Dow Corning 200 Fluid | Dimethicone | 2.00 |
| | (300 cs) (Dow Corning) | | |
| | Betone Gel Mio | Mineral oil, | 3.00 |
| | (Elementis) | quaternium-18 hectorite, | |
| | | propylene carbonate | |
| B | Demineralized water | Water (aqua) | ad 100 |
| | Magnesium sulfate | Magnesium sulfate | 1.00 |
| | heptahydrate (Merck) | | |
| | Glycerin, 99.5% | Glycerin | 3.00 |
| | Drago-Beta-Glucan | Water (aqua), butylene | 5.00 |
| | (Symrise) | glycol, glycerin, | |
| | | Avena sativa (oat) | |
| | | kernel extract | |
| | Raspberry leaf extract | | 0.005 |
| | Aloe extract | | 0.450 |
| | Magnesium ascorbyl | | 0.045 |
| | phosphate | | |
| | Dragocid Liquid (Symrise) | Phenoxyethanol, | 0.80 |
| | | methylparaben, | |
| | | ethylparaben, | |
| | | butylparaben, | |
| | | propylparaben, | |
| | | isobutylparaben | |
| C | Symrise perfume oil | Fragrance | 0.30 |

Heat parts A and B to 75° C. without Drago-Beta-Glucan, Add Drago-Beta-Glucan to part B. Add part B to part A and emulsify. Stir emulsion until cold, homogenize once more at approx, 50° C. and add perfume oil at approx. 35° C.

### 4.2 Night Cream Against Skin Ageing

| | Raw material name | | Content in wt. |
|---|---|---|---|
| Part | (manufacturer) | INCI name | % |
| A | Dragosan W/O P (Symrise) | Sorbitan isostearate, | 6.00 |
| | | hydrogenated castor oil, | |
| | | ceresin, beeswax (cera alba) | |
| | PCL Liquid (Symrise) | Cetearyl ethylhexanoate, | 12.00 |
| | | isopropyl myristate | |
| | Sunflower oil (H. Erhard | Helianthus annuus (sunflower) | 5.00 |
| | Wagner) | seed oil | |
| | Sweet almond oil (H. Erhard | Prunus dulcis | 5.00 |
| | Wagner) | | |
| | Dragosan W/O Liquid | Polyglyceryl-3 polyricinoleate, | 1.00 |
| | (Symrise) | sorbitan isostearate | |
| | Alugel 34 TH (Baerlocher) | Aluminium stearate | 1.00 |
| | Oxynex 2004 (Merck) | BHT | 0.10 |
| B | Demineralized water | Water (aqua) | ad 100 |
| | Glycerin, 99.5% | Glycerin | 2.00 |
| | Karion F (Merck) | Sorbitol | 2.00 |
| | Aloe Vera Gel Concentrate | Water (aqua), Aloe barbadensis | 3.00 |
| | 10/1 (Symrise) | leaf juice | |
| | Extrapon witch hazel | Propylene glycol, Hamamelis | 1.00 |
| | distillate colorless (Symrise) | virginiana (witch hazel) water, | |
| | | water (aqua), Hamamelis | |
| | | virginiana (witch hazel) extract | |
| | Strawberry leaf extract | | 0.040 |
| | Aloe extract | | 2.000 |
| | Sodium ascorbyl | | 0.400 |
| | phosphate | | |
| | Magnesium sulfate | Magnesium sulfate | 0.70 |
| | heptahydrate (Merck) | | |
| | Dragocid Liquid (Symrise) | Phenoxyethanol, | 0.80 |
| | | methylparaben, ethylparaben, | |
| | | butylparaben, propylparaben, | |
| | | isobutylparaben | |
| C | Vitamin E acetate (DSM | Tocopheryl acetate | 3.00 |
| | Nutritional Products) | | |
| | Vitamin A palmitate in oil (1 | Retinyl palmitate | 0.20 |
| | million IU/g) (DSM | | |
| | Nutritional Products) | | |
| | -(-alpha-)-Bisabolol, natural | Bisabolol | 0.10 |
| | (Symrise) | | |
| | Symrise perfume oil | Fragrance | 0.40 |

Heat parts A and B separately to approx. 80° C. Add part B to part A, emulsify and stir until cold. At approx. 60° C., homogenize once more and add part C at approx. 35° C.

4.3 O/W Lotion Against Skin Ageing with UVA/B Broadband Protection

| | Raw material name | | Content in wt. |
|---|---|---|---|
| Part | (manufacturer) | INCI name | % |
| A | Emulsiphos (Symrise) | Potassium cetyl phosphate, | 1.50 |
| | | hydrogenated palm glycerides | |
| | Tegosoft TN (Degussa) | C12-15 alkyl benzoate | 5.00 |
| | Copherol 1250 (Cognis) | Tocopheryl acetate | 0.50 |
| | Lanette O (Cognis) | Cetearyl alcohol | 1.00 |
| | Neutral oil (Symrise) | Caprylic/capric triglyceride | 2.00 |
| | Dow Corning 246 Fluid (Dow | Cyclohexasiloxane (and) | 2.00 |
| | Corning) | cyclopentasiloxane | |
| | Neo Heliopan AV (Symrise) | Ethylhexyl methoxycinnamate | 3.00 |
| | Neo Heliopan OS (Symrise) | Ethylhexyl salicylate | 5.00 |
| | Neo Heliopan MBC | 4-Methylbenzylidene camphor | 1.50 |
| | (Symrise) | | |
| | Neo Heliopan 357 (Symrise) | Butyl methoxydibenzoylmethane | 1.00 |
| | EDETA DB (BASF) | Disodium EDTA | 0.10 |
| | Keltrol T (Danby-Chemie) | Xanthan gum | 0.20 |
| | Carbopol ETD 2050 | Carbomer | 0.20 |
| | (Noveon) | | |
| B | Demineralized water | Water (aqua) | ad 100 |
| | Glycerin, 99.5% | Glycerin | 4.70 |
| | Dragocid liquid (Symrise) | Phenoxyethanol, | 0.70 |
| | | methylparaben, ethylparaben, | |
| | | butylparaben, propylparaben, | |
| | | isobutylparaben | |
| | Neo Heliopan ® AP (22% aq. | Disodium phenyl | 4.55 |
| | solution neutralized with | dibenzimidazole tetrasulfonate | |
| | triethanolamine) (Symrise) | | |
| | Neo Heliopan Hydro (30% | Phenylbenzimidazole sulfonic | 6.67 |
| | aq. solution neutralized with | acid | |
| | triethanolamine) (Symrise) | | |
| | Strawberry leaf extract | | 0.10 |
| | Aloe extract | | 1.80 |
| | Magnesium ascorbyl | | 0.60 |
| | phosphate | | |
| C | Triethanolamine, 99% | Triethanolamine | 0.50 |
| D | Symrise perfume oil | Fragrance | 0.40 |
| | Dragosantol (Symrise) | Bisabolol | 0.10 |

Heat part A (apart from Keltrol and Carbopol) to 85° C. Add Keltrot and Carbopol and homogenize. Heat part B to 85° C. and add part B to part A. Add part C directly to part A/B, homogenize and leave to cool. Add part D to part A/B/C and homogenize. The pH value of the final product should be approx. 7.2 to 7.5.

### 4.4 Aftersun Lotion (O/W) Against Skin Ageing

| | Raw material name | | Content in wt. |
|---|---|---|---|
| Part | (manufacturer) | INCI name | % |
| A | Demineralized water | Water (aqua) | ad 100 |
| | Glycerin, 99.5% | Glycerin | 4.00 |
| | D-Panthenol (BASF) | Panthenol | 1.00 |
| | Butylene glycol | Butylene glycol | 5.00 |
| | Allantoin (Merck) | Allantoin | 0.10 |
| | Aloe Vera Gel Concentrate | Water (aqua), Aloe barbadensis | 3.0 |
| | 10/1 (Symrise) | leaf juice | |
| | Raspberry leaf extract | | 0.10 |
| | Aloe extract | | 1.50 |
| | Sodium ascorbyl | | 0.50 |
| | phosphate | | |
| | Lara Care A-200 (Rahn) | Galactoarabinan | 0.25 |
| B | Baysilone Oil M 350 (GE | Dimethicone | 1.00 |
| | Bayer Silicones) | | |
| | Copherol 1250 (Cognis) | Tocopheryl acetate | 0.50 |
| | Tegosoft TN (Degussa) | C12-15 alkyl benzoate | 5.00 |
| | Cetiol SB 45 (Cognis) | Butyrospermum parkii (Shea | 1.00 |
| | | butter) | |
| | Cetiol OE (Cognis) | Dicaprylyl ether | 4.00 |
| | -(-alpha-)-Bisabolol, natural | Bisabolol | 0.10 |
| | (Symrise) | | |
| | Dragocid Liquid (Symrise) | Phenoxyethanol, | 0.70 |
| | | methylparaben, ethylparaben, | |
| | | butylparaben, propylparaben, | |
| | | isobutylparaben | |
| | Pemulen TR-2 (Noveon) | Acrylates/C10-30 alkyl acrylate | 0.25 |
| | | crosspolymer | |
| | Frescolat ML cryst. (Symrise) | Menthyl lactate | 0.80 |
| | Symrise perfume oil | Fragrance | 0.30 |
| C | Sodium hydroxide (10% aq. | Sodium hydroxide | 0.60 |
| | solution) | | |

Dissolve part A in water. Dissolve Frescolat ML cryst. and Cetiol SB 45 from part B with heating to at most 35° C. in Tegosoft Tenn., leave to cool and add the remaining constituents of part B. Add part B to part A with stirring and homogenize. The pH value of the final product should be approx. 6.0.

### 4.5 Bodyspray O/W Against Skin Ageing

| | Raw material name | | Content |
|---|---|---|---|
| Part | (manufacturer) | INCI name | in wt. % |
| A | Dracorin GOC (Symrise) | Glyceryl oleate citrate, | 2.00 |
| | | caprylic/capric triglyceride | |
| | Neutral oil | Caprylic/capric triglyceride | 4.00 |
| | Paraffin oil 5 E | Paraffinum liquidum | 4.00 |
| | (Parafluid) | | |
| | PCL Liquid 100 | Cetearyl ethylhexanoate | 7.00 |
| | (Symrise) | | |
| | Dragoxat EH (Symrise) | Ethylhexyl ethylhexanoate | 4.00 |
| | Dow Corning 345 Fluid | Cyclomethicone | 0.50 |
| | (Dow Corning) | | |
| | Dragosantol (Symrise) | Bisabolol | 0.10 |
| | Symrise perfume oil | Fragrance | 0.20 |
| B | Demineralized water | Water (aqua) | ad 100 |
| | Pemulen TR-2 (Noveon) | Acrylates/C10-30 alkyl | 0.20 |
| | | acrylate crosspolymer | |
| | Hydrolite-5 (Symrise) | Pentylene glycol | 5.00 |
| | Drago-Oat-Active | Water (aqua), butylene | 1.00 |
| | (Symrise) | glycol, Avena sativa (oat) | |
| | | kernel extract | |
| | Raspberry leaf extract | | 0.001 |
| | Aloe extract | | 0.990 |
| | Sodium ascorbyl | | 0.020 |
| | phosphate | | |
| C | Sodium hydroxide | Sodium hydroxide | 0.40 |
| | (10% aq. solution) | | |

Swell Pemulen in water, then add the remaining raw materials of part B. Mix part A. Add part B to part A without stirring and only then emulsify. Add part C during emulsification. The pH value of the final product should be approx. 6.3.

### 4.6 O/W Cream Against Skin Ageing

| | Raw material name | | Content |
|---|---|---|---|
| Part | (manufacturer) | INCI name | in wt. % |
| A | Dracorin GMS (Symrise) | Glyceryl stearate | 2.00 |
| | PCL Solid (Symrise) | Stearyl heptanoate, stearyl | 2.00 |
| | | caprylate | |
| | Lanette O (Cognis) | Cetearyl alcohol | 3.00 |
| | PCL Liquid 100 | Cetearyl ethylhexanoate | 5.00 |
| | (Symrise) | | |
| | Isodragol (Symrise) | Triisononanoin | 2.00 |
| | Abil 350 (Degussa- | Dimethicone | 2.00 |
| | Goldschmidt) | | |
| | Dragoxat EH (Symrise) | Ethylhexyl ethylhexanoate | 3.00 |
| B | Demineralized water | Water (aqua) | ad 100 |
| | Carbopol Ultrez-10 | Carbomer | 0.10 |
| | (Noveon) | | |
| | Keltrol RD (CP-Kelco) | Xanthan gum | 0.10 |
| | Emulsiphos (Symrise) | Potassium cetyl phosphate, | 2.00 |
| | | hydrogenated palm | |
| | | glycerides | |
| | Dragocid Liquid | Phenoxyethanol, | 0.80 |
| | (Symrise) | methylparaben, | |
| | | ethylparaben, | |
| | | butylparaben, | |
| | | propylparaben, | |
| | | isobutylparaben | |
| | Extrapon Chamomile | Glycerin, water (aqua), | 0.50 |
| | GW (Symrise) | Chamomilla recutita | |
| | | (matricaria) flower extract | |
| | Extrapon Rosemary GW | Glycerin, water (aqua), | 0.30 |
| | (Symrise) | Rosmarinus officinalis | |
| | | (rosemary) leaf extract | |
| | Extrapon Green Tea GW | Glycerin, water (aqua), | 0.20 |
| | (Symrise) | Camellia sinensis | |
| | | leaf extract | |
| | Meadowsweet herb | | 0.960 |
| | extract | | |
| | Aloe extract | | 0.030 |
| | Magnesium ascorbyl | | 0.003 |
| | phosphate | | |
| | 1,2-Propylene glycol 99P | Propylene glycol | 5.00 |
| | GC | | |
| | Glycerin 85P | Glycerin | 2.00 |
| C | Sodium hydroxide | Sodium hydroxide | 0.25 |
| | (10% aq. solution) | | |
| D | Symrise perfume oil | Fragrance | 0.30 |

Preswell Carbopol Ultrez 10 and Keltrol RD in the water and add the remaining raw materials of part B thereto. Heat parts A and B separately to approx. 80° C. Add part A to part B and emulsify while adding part C. Stir until cold and add part D at approx. 35° C. The pH value of the final product should be approx. 5.5.

### 4.7 Shampoo Against Skin Ageing

| | Raw material name | | Content in |
|---|---|---|---|
| Part | (manufacturer) | INCI name | wt. % |
| A | Genapol LRO liquid (Cognis) | Sodium laureth sulfate | 37.00 |
| | Dragoderm (Symrise) | Glycerin, Triticum | 2.00 |
| | | vulgare (wheat) | |
| | | gluten, water (aqua) | |
| | Raspberry leaf extract | | 0.01 |
| | Aloe extract | | 0.9 |
| | Magnesium ascorbyl | | 0.09 |
| | phosphate | | |
| B | Demineralized water | Water (aqua) | ad 100 |
| | Merquat 550 (Ondeo Nalco) | Polyquaternium-7 | 0.50 |
| C | Demineralized water | Water (aqua) | 20.00 |
| | Comperlan 100 (Cognis) | Cocamide MEA | 0.50 |
| D | Tego Betaine L7 unpreserved | Cocamidopropyl | 6.00 |
| | (Degussa-Goldschmidt) | betaine | |
| | Citric acid 10% | Citric acid | 0.30 |
| | EDETA B powder (BASF) | Tetrasodium EDTA | 0.10 |
| | Sodium benzoate | Sodium benzoate | 0.50 |
| | Sodium chloride | Sodium chloride | 1.00 |
| | Symrise perfume oil | Fragrance | 0.50 |

Dissolve Dragoderm and blackberry leaf extract in Genapol LRO. Predissolve Merquat 550 in water and add. Dissolve part C with stirring and heating and leave to cool. Dissolve part C in part A/B. Add the raw materials of part D in succession and stir. The pH value of the final product should be approx. 5.0.

### 4.8 O/W Cream Against Skin Ageing

| | Raw material name | | Content |
|---|---|---|---|
| Part | (manufacturer) | INCI name | in wt. % |
| A | Dracorin CE (Symrise) | Glyceryl stearate citrate | 5.00 |
| | Neutral oil (Symrise) | Caprylic/capric triglyceride | 6.00 |
| | Isopropyl palmitate | Isopropyl palmitate | 4.00 |
| | (Symrise) | | |
| | Lanette 16 (Cognis) | Cetyl alcohol | 1.00 |
| | PCL Liquid 100 | Cetearyl ethylhexanoate | 3.00 |
| | (Symrise) | | |
| | Dragoxat EH (Symrise) | Ethylhexyl ethylhexanoate | 3.00 |
| | Abil 350 (Degussa- | Dimethicone | 0.50 |
| | Goldschmidt) | | |
| B | Demineralized water | Water (aqua) | ad 100 |
| | Keltrol RD (Rahn) | Xanthan gum | 0.20 |
| | Symdiol 68 (Symrise) | 1,2-Hexanediol, caprylyl | 0.50 |
| | | glycol | |
| | Drago-Beta-Glucan | Water (aqua), butylene | 0.30 |
| | (Symrise) | glycol, glycerin, Avena | |
| | | sativa (oat) kernel | |
| | | extract | |
| | Blackberry leaf extract | | 0.02 |
| | Aloe extract | | 1.80 |
| | Sodium ascorbyl | | 0.18 |
| | phosphate | | |
| | Glycerin 85P | Glycerin | 3.00 |
| C | Symrise perfume oil | Fragrance | 0.30 |

Swell Keltrol in water and add the remaining raw materials of part B to Drago-Beta-Glucan. Heat parts A and B separately to approx. 80° C. Add Drago-Beta-Glucan to part B. Add part B to part A and only then emulsify. Stir until cold and add part C at approx. 35° C. The pH value of the final product should be approx. 5.5.

### EXAMPLE 5

### Further Examples of Application in the Field of Oral Hygiene

The oral hygiene products containing synergistically active ternary leaf extract according to the invention are applied by being introduced in a sufficient amount into the oral cavity in the conventional manner in combination with other active ingredients and additives. Advantageous preparations for some applications are stated by way of example below. Unless otherwise stated, all stated values relate to weight.

5.1. Gel Tooth Cream with Activity Against Bad Breath

| | I (%) | II (%) | III (%) |
|---|---|---|---|
| Na carboxymethylcellulose | 0.40 | 0.40 | 0.40 |
| Sorbitol 70%, in water | 72.00 | 72.00 | 72.00 |
| Polyethylene glycol (PEG) 1500 | 3.00 | 3.00 | 3.00 |
| Na saccharinate | 0.07 | 0.07 | 0.07 |
| Na fluoride | 0.24 | 0.24 | 0.24 |
| p-Hydroxybenzoic acid (PHB) ethyl | 0.15 | 0.15 | 0.15 |
| ester | | | |
| Aroma A (see below) | 0.10 | 0.80 | 0.75 |
| Ternary mixture of Aloe extract, | 0.01 | 0.40 | 1.00 |
| sodium ascorbyl phosphate and | | | |
| raspberry leaf extract (quantity ratio: | | | |
| 90:9:1 w/w/w) | | | |
| Abrasive silica | 11.00 | 11.00 | 11.00 |
| Thickening silica | 6.00 | 6.00 | 6.00 |
| Sodium dodecyl sulfate (SDS) | 1.40 | 1.40 | 1.40 |
| Water, dist. | ad 100.00 | ad 100.00 | ad 100.00 |

Aroma A was of the following composition:

30 wt. % I-menthol, 30 wt. % peppermint oil Mentha piperita, 21.5 wt. % peppermint oil Mentha arvensis, 9 wt. % anethole, 0.5 wt. % anisaldehyde, 2 wt. % eucalyptol, 1 wt. % Eucalyptus globulus oil, 3 wt. % menthone, 1 wt. % spearmint oil, 1 wt. % basil oil, 0.5 wt. % menthyl acetate, 0.05 wt. % menthyl lactate, 0.1 wt. % menthyl-3-carboxylic acid-N-ethylamide (WS-3), 0.06 wt. % 2-hydroxyethylmenthyl carbonate (Frescolat MGC, Symrise), 0.05 wt. % 2-hydroxypropylmenthyl carbonate, (Frescolat MPC, Symrise), 0.1 wt. % pinene, 0.1 wt. % propylene glycol, 0.05 wt. % limonene.
5.2. Tooth Cream with Activity Against Plaque

| | I (%) | II (%) | III (%) |
|---|---|---|---|
| Na carboxymethylcellulose | 1.00 | 1.00 | 1.00 |
| Glycerin | 12.50 | 12.50 | 12.50 |
| Sorbitol 70%, in water | 29.00 | 29.00 | 29.00 |
| Na saccharinate | 0.20 | 0.20 | 0.20 |
| Na fluoride | 0.22 | 0.22 | 0.22 |
| Azacycloheptane-2,2-diphosphoric | 1.00 | 1.00 | 1.00 |
| acid, disodium salt | | | |
| Bromochlorophene | 0.10 | 0.10 | 0.10 |
| Peppermint aroma | 0.05 | 1.10 | 0.35 |
| Ternary mixture of Aloe extract, | 1.05 | 1.30 | 1.70 |
| magnesium ascorbyl phosphate and | | | |
| raspberry leaf extract (quantity ratio: | | | |
| 90:9:1 w/w/w) | | | |
| Chamomile extract (Cremogen Forte | 0.20 | 0.50 | 0.10 |
| Chamomile, Symrise) | | | |
| Sage extract (Extrapon Sage GW, | 0.20 | 0.10 | 0.30 |
| Symrise) | | | |
| Abrasive silica | 14.00 | 14.00 | 14.00 |
| Thickening silica | 5.00 | 5.00 | 5.00 |
| Sodium dodecyl sulfate (SDS | 1.50 | 1.50 | 1.50 |
| Water, dist. | ad 100.00 | ad 100.00 | ad 100.00 |

5.3. Tooth Cream with Activity Against Plaque

Base: silica, alkali metal diphosphate

| | I (%) | II (%) | III (%) |
|---|---|---|---|
| Carragenan | 0.90 | 0.90 | 0.90 |
| Glycerin | 15.00 | 15.00 | 15.00 |
| Sorbitol 70%, in water | 25.00 | 25.00 | 25.00 |
| PEG 1000 | 3.00 | 3.00 | 3.00 |
| Na fluoride | 0.24 | 0.24 | 0.24 |
| Tetrapotassium diphosphate | 4.50 | 4.50 | 4.50 |
| Tetrasodium diphosphate | 1.50 | 1.50 | 1.50 |
| Na saccharinate | 0.40 | 0.40 | 0.40 |
| Precipitated silica | 20.00 | 20.00 | 20.00 |
| Titanium dioxide | 1.00 | 1.00 | 1.00 |
| PHB methyl ester | 0.10 | 0.10 | 0.10 |
| Menthol/eucalyptol aroma | 1.10 | 0.80 | 0.20 |
| Ternary mixture of Aloe | 0.10 | 0.40 | 1.00 |
| extract, sodium ascorbyl | | | |
| phosphate and blackberry | | | |
| leaf extract (quantity ratio: | | | |
| 90:9:1 w/w/w) | | | |
| Sodium dodecyl sulfate | 1.30 | 1.30 | 1.30 |
| Water, dist. | ad 100.00 | ad 100.00 | ad 100.00 |

### 5.4. Tooth Cream for Sensitive Teeth

| | I (%) | II (%) | III (%) |
|---|---|---|---|
| Na carboxymethylcellulose | 0.70 | 0.70 | 0.70 |
| Xanthan Gum | 0.50 | 0.50 | 0.50 |
| Glycerin | 15.00 | 15.00 | 15.00 |
| Sorbitol 70%, in water | 12.00 | 12.00 | 12.00 |
| K nitrate | 5.00 | 5.00 | 5.00 |
| Na monofluorophosphate | 0.80 | 0.80 | 0.80 |
| PHB methyl ester | 0.15 | 0.15 | 0.15 |
| PHB propyl ester | 0.05 | 0.05 | 0.05 |
| Na saccharinate | 0.20 | 0.20 | 0.20 |
| Menthol/anethole aroma | 0.25 | 0.75 | 0.25 |
| Ternary mixture of Aloe | 0.02 | 1.00 | 1.50 |
| extract, sodium ascorbyl | | | |
| phosphate and raspberry leaf | | | |
| extract (quantity ratio: 90:9:1 | | | |
| w/w/w) | | | |
| Rosemary extract (Extrapon | 0.20 | 0.01 | 0.10 |
| Rosemary, Symrise) | | | |
| Ca carbonate | 35.00 | 35.00 | 35.00 |
| Silicon dioxide | 1.00 | 1.00 | 1.00 |
| Sodium dodecyl sulfate | 1.50 | 1.50 | 1.50 |
| (SDS) | | | |
| Water, dist. | ad 100.00 | ad 100.00 | ad 100.00 |

### 5.5. Tooth Cream for Sensitive Teeth

| | I (%) | II (%) | III (%) |
|---|---|---|---|
| Hydroxyethylcellulose | 1.40 | 1.40 | 1.40 |
| Guar gum | 0.60 | 0.60 | 0.60 |
| Glycerin | 18.00 | 18.00 | 18.00 |
| Sorbitol 70%, in water | 12.00 | 12.00 | 12.00 |
| Na saccharinate | 0.35 | 0.35 | 0.35 |
| Dye | 0.01 | 0.01 | 0.01 |
| PHB methyl ester | 0.15 | 0.15 | 0.15 |
| PHB propyl ester | 0.04 | 0.04 | 0.04 |
| Sr chloride | 10.50 | 10.50 | 10.50 |
| Peppermint/aniseed | 0.35 | 1.20 | 0.60 |
| aroma | | | |
| Ternary mixture of Aloe | 0.05 | 0.50 | 0.90 |
| extract, magnesium | | | |
| ascorbyl phosphate and | | | |
| blackberry leaf extract | | | |
| (quantity ratio: 90:9:1 | | | |
| w/w/w) | | | |
| Green tea extract | 0.25 | 0.10 | 0.05 |
| (Extrapon Green Tea GW, | | | |
| Symrise) | | | |
| Precipitated silica | 15.00 | 15.00 | 15.00 |
| Silicon dioxide | 1.60 | 1.60 | 1.60 |
| Sodium dodecyl sulfate | 1.30 | 1.30 | 1.30 |
| Water, dist. | ad 100.00 | ad 100.00 | ad 100.00 |

5.6. Ready-to-Use Mouthwash with Fluoride

| | I (%) | II (%) | III (%) |
|---|---|---|---|
| Ethanol | 7.00 | 7.00 | 7.00 |
| Glycerin | 12.00 | 12.00 | 12.00 |
| Na fluoride | 0.05 | 0.05 | 0.05 |
| Pluronic F-127 ® (BASF, | 1.40 | 1.40 | 1.40 |
| surface-active substance) | | | |
| Na phosphate buffer pH 7.0 | 1.10 | 1.10 | 1.10 |
| Sorbic acid | 0.20 | 0.20 | 0.20 |
| Na saccharinate | 0.10 | 0.10 | 0.10 |
| Menthol/peppermint aroma | 0.08 | 0.20 | 0.15 |
| Ternary mixture of Aloe | 0.02 | 0.70 | 0.10 |
| extract, magnesium | | | |
| ascorbyl phosphate and | | | |
| strawberry leaf extract | | | |
| (quantity ratio: 96.5:3.2:0.3 | | | |
| w/w/w) | | | |
| Bisabolol | 0.01 | 0.05 | 0.20 |
| Melissa extract (Extrapon | 0.30 | 0.50 | 0.05 |
| Melissa, Symrise) | | | |
| Sage extract (Extrapon | 0.30 | 0.10 | 0.05 |
| Sage, Symrise) | | | |
| Dye | 0.01 | 0.01 | 0.01 |
| Water, dist. | ad 100.00 | ad 100.00 | ad 100.00 |

### 5.7. Mouthwash Concentrate

| | I (%) | II (%) | III (%) |
|---|---|---|---|
| Ethanol, 95% | 80.00 | 80.00 | 80.00 |
| Na cyclamate | 0.15 | 0.15 | 0.15 |
| Menthol/aniseed/ | 1.50 | 2.00 | 2.00 |
| eucalyptol aroma | | | |
| Dye | 0.01 | 0.01 | 0.01 |
| Ternary mixture of Aloe | 1.50 | 2.50 | 5.00 |
| extract, sodium ascorbyl | | | |
| phosphate and | | | |
| meadowsweet herb | | | |
| extract (quantity ratio: | | | |
| 90:9:1 w/w/w) | | | |
| Green tea extract | 0.20 | 1.00 | 0.50 |
| (Extrapon Green Tea GW, | | | |
| Symrise) | | | |
| Bisabolol | 0.50 | 0.20 | 1.00 |
| Water, dist. | ad 100.00 | ad 100.00 | ad 100.00 |

### 5.8. Chewing Gum

| | I (%) | II (%) | III (%) |
|---|---|---|---|
| Chewing gum base | 21.00 | 21.00 | 21.00 |
| Glucose syrup | 16.50 | 16.50 | 16.50 |
| Glycerin | 0.50 | 0.50 | 0.50 |
| Confectioners' sugar | 60.30 | 60.00 | 59.80 |
| Menthol/spearmint aroma | 1.20 | 1.00 | 0.70 |
| Ternary mixture of Aloe | 0.50 | 1.00 | 1.50 |
| extract, magnesium | | | |
| ascorbyl phosphate and | | | |
| strawberry leaf extract | | | |
| (quantity ratio: 90:9:1 | | | |
| w/w/w) | | | |

### 5.9. Sugar-Free Chewing Gum

| | I (%) | II (%) | III (%) |
|---|---|---|---|
| Chewing gum base | 30.00 | 30.00 | 30.00 |
| Sorbitol, powder | 38.45 | 38.40 | 38.30 |
| Palatinite | 9.50 | 9.50 | 9.50 |
| Xylitol | 2.00 | 2.00 | 2.00 |
| Mannitol | 3.00 | 3.00 | 3.00 |
| Aspartame | 0.10 | 0.10 | 0.10 |
| Acesulfame ® K | 0.10 | 0.10 | 0.10 |
| Emulgum/emulsifier | 0.30 | 0.30 | 0.30 |
| Sorbitol 70%, in water | 14.00 | 14.00 | 14.00 |
| Glycerin | 1.00 | 1.00 | 1.00 |
| Menthol/aniseed/ | 1.20 | 0.80 | 0.60 |
| cinnamon aroma | | | |
| Ternary mixture of Aloe | 0.35 | 0.80 | 1.10 |
| extract, sodium ascorbyl | | | |
| phosphate and raspberry | | | |
| leaf extract (quantity | | | |
| ratio: 96.5:3.2:0.3 w/w/w) | | | |

### 5.10. Gelatine Capsules for Direct Consumption

| | I (%) | II (%) | III (%) |
|---|---|---|---|
| Gelatin shell: | | | |
| Glycerin | 2.014 | 2.014 | 2.014 |
| Gelatin 240 Bloom | 7.91 | 7.91 | 7.91 |
| Sucralose | 0.065 | 0.065 | 0.065 |
| Allura Red | 0.006 | 0.006 | 0.006 |
| Brilliant blue | 0.005 | 0.005 | 0.005 |
| Core composition: | | | |
| Vegetable oil triglyceride | 80.0 | 75.0 | 77.5 |
| (coconut oil fraction) | | | |
| Aroma B | 9.95 | 12.0 | 12.0 |
| Ternary mixture of Aloe | 0.05 | 3.0 | 0.5 |
| extract, magnesium | | | |
| ascorbyl phosphate and | | | |
| raspberry leaf extract | | | |
| (quantity ratio: 90:9:1 | | | |
| w/w/w) | | | |

Aroma B was of the following composition (values in each case in wt. %):

0.1% neotame powder, 0.05% aspartame, 29.3% peppermint arvensis oil, 29.3% peppermint piperita oil Willamette, 2.97% sucralose, 2.28% triacetin, 5.4% diethyl tartrate, 12.1% peppermint oil yakima, 0.7% ethanol, 3.36% 2-hydroxyethylmenthyl carbonate, 3.0% 2-hydroxypropylmenthyl carbonate, 0.27% vanillin, 5.5% d-limonene, 5.67% L-menthyl acetate.

The gelatine capsules suitable for direct consumption (production in a manner similar to WO 2004/050069) had a diameter of 5 mm, the weight ratio of core material to shell material was 90:10. The capsule opened in the mouth within less than 10 seconds and dissolved completely within less than 50 seconds.

### EXAMPLES 6

### Further Formulations Comprising Preparations According to the Invention with a Collagen Synthesis Stimulating Activity

| RAW MATERIAL | | | | | | | | | | | |
|---|---|---|---|---|---|---|---|---|---|---|---|
| NAME | | WEIGHT % | | | | | | | | | |
| (MANUFACTURER) | INCI | 6.1 | 6.2 | 6.3 | 6.4 | 6.5 | 6.6 | 6.7 | 6.8 | 6.9 | 6.10 |
| Aloe extract | | 0.9 | 1.8 | 0.9 | 2.0 | 0.8 | 1.5 | 2.7 | 2.0 | 0.6 | 1.0 |
| Sodium ascorbyl | | 0.09 | | 0.09 | 0.5 | | 0.15 | | 0.5 | | 0.2 |
| phosphate | | | | | | | | | | | |
| Magnesium | | | 0.18 | | | 0.3 | | 0.27 | | 0.06 | |
| ascorbyl | | | | | | | | | | | |
| phosphate | | | | | | | | | | | |
| Raspberry leaf | | 0.01 | | | 0.1 | 0.1 | 0.05 | | | | 0.02 |
| extract | | | | | | | | | | | |
| Meadowsweet | | | 0.02 | | | | | | 0.1 | | |
| herb extract | | | | | | | | | | | |
| Strawberry leaf | | | | | | | | 0.03 | | 0.01 | |
| extract | | | | | | | | | | | |
| Blackberry leaf | | | | 0.01 | | | | | | | |
| extract | | | | | | | | | | | |
| -(-Alpha-)- | Bisabolol | 0.3 | 0.4 | 0.3 | 0.1 | 0.3 | 0.2 | 0.05 | 0.2 | 0.5 | 0.1 |
| Bisabolol, natural | | | | | | | | | | | |
| (Symrise) | | | | | | | | | | | |
| Ginger CO₂ | Zingiber | 0.003 | 0.004 | 0.003 | 0.005 | 0.003 | 0.002 | 0.001 | 0.002 | 0.01 | 0.001 |
| extract (Flavex) | officinale | | | | | | | | | | |
| | (ginger) root | | | | | | | | | | |
| | extract | | | | | | | | | | |
| Abil 350 | Dimethicone | 0.5 | 2.0 | 1.0 | | | | | 0.5 | 0.5 | |
| (Degussa- | | | | | | | | | | | |
| Goldschmidt) | | | | | | | | | | | |
| Allantoin (Merck) | Allantoin | | 0.2 | 0.1 | | | | | | | |
| Aloe Vera Gel | Water (aqua), | | | 3.0 | | | 3.0 | | | | |
| Concentrate 10/1 | Aloe | | | | | | | | | | |
| (Symrise) | barbadensis leaf | | | | | | | | | | |
| | juice | | | | | | | | | | |
| Alugel 34 TH | Aluminium | | | | | | 1.0 | | | | |
| (Baerlocher) | stearate | | | | | | | | | | |
| Aqua-Ceramide | Cetyloxypropyl | | 0.1 | | | | | | | | 0.1 |
| (Kao) | glyceryl | | | | | | | | | | |
| | methoxypropyl | | | | | | | | | | |
| | myristamide | | | | | | | | | | |
| Arbutin (Sabinsa) | β-Arbutin | 1.0 | | | | | | | | | |
| Sodium ascorbyl | Sodium ascorbyl | 2.0 | | 1.0 | | | | | | | |
| phosphate (EMD | phosphate | | | | | | | | | | |
| Chemicals) | | | | | | | | | | | |
| Butylene glycol | Butylene glycol | | | 5.0 | | | | | | | |
| Carbopol ETD | Carbomer | | | | | 0.2 | | | | | |
| 2050 (Noveon) | | | | | | | | | | | |
| Carbopol Ultrez-10 | Carbomer | | 0.1 | | | | | | | | |
| (Noveon) | | | | | | | | | | | |
| Ceramide 2 | Ceramide 2 | 0.1 | | | | | | | | | |
| (Sederma) | | | | | | | | | | | |
| Ceramide PC104 | Hydroxypropyl | | | | | 0.1 | | | | | |
| (Pacific | bispalmitamide | | | | | | | | | | |
| Corporation) | MEA | | | | | | | | | | |
| Ceramide SL | Hydroxyethyl | | | | | | 0.1 | | | | |
| (Sino Lion) | palmityl | | | | | | | | | | |
| | oxyhydroxy- | | | | | | | | | | |
| | propyl palmit- | | | | | | | | | | |
| | amide | | | | | | | | | | |
| Cetiol OE (Cognis) | Dicaprylyl ether | | | 4.0 | | | | | | | |
| Cetiol SB 45 | Butyrospermum | | | 1.0 | | | | | | | |
| (Cognis) | parkii (shea | | | | | | | | | | |
| | butter) | | | | | | | | | | |
| Citric acid 10% | Citric acid | | | | | | | 0.3 | | | |
| sol. | | | | | | | | | | | |
| Comperlan 100 | Cocamide MEA | | | | | | | 0.5 | | | |
| (Cognis) | | | | | | | | | | | |
| Dihydroxyacetone | Dihydroxyacetone | | | | | | | | 5.0 | | |
| (Merck) | | | | | | | | | | | |
| Dow Corning 246 | Cyclohexa- | | | | | 2.0 | | | | | |
| Fluid (Dow | siloxane and | | | | | | | | | | |
| Corning) | cyclopenta- | | | | | | | | | | |
| | siloxane | | | | | | | | | | |
| Dow Corning 345 | Cyclomethicone | | | | 0.5 | | | | | | |
| Fluid (Dow | | | | | | | | | | | |
| Corning) | | | | | | | | | | | |
| D-Panthenol | Panthenol | | | 1.0 | | | | | | | |
| (BASF) | | | | | | | | | | | |
| Dracorin CE | Glyceryl stearate | 5.0 | | | | | | | 5.0 | 1.5 | |
| (Symrise) | citrate | | | | | | | | | | |
| Dracorin GMS | Glyceryl stearate | | 2.0 | | | | | | | 2.0 | |
| (Symrise) | | | | | | | | | | | |
| Dracorin GOC | Glyceryl oleate | | | | 2.0 | | | | | | |
| (Symrise) | citrate, | | | | | | | | | | |
| | caprylic/capric | | | | | | | | | | |
| | triglyceride | | | | | | | | | | |
| Drago-Beta- | Water (aqua), | 0.3 | | | | | | | | | |
| Glucan (Symrise) | butylene glycol, | | | | | | | | | | |
| | glycerin, Avena | | | | | | | | | | |
| | sativa (oat), | | | | | | | | | | |
| | kernel extract | | | | | | | | | | |
| Dragocid Liquid | Phenoxyethanol, | | 0.8 | 0.7 | | 0.7 | 0.8 | | | 0.8 | |
| (Symrise) | methylparaben, | | | | | | | | | | |
| | ethylparaben, | | | | | | | | | | |
| | butylparaben, | | | | | | | | | | |
| | propylparaben, | | | | | | | | | | |
| | isobutylparaben | | | | | | | | | | |
| Dragoderm | Glycerin, | | | | | | | 2.0 | | | |
| (Symrise) | Triticum vulgare | | | | | | | | | | |
| | (wheat) gluten, | | | | | | | | | | |
| | water (aqua) | | | | | | | | | | |
| Drago-Oat-Active | Water (aqua), | | | | 1.0 | | | | | | |
| (Symrise) | butylene glycol, | | | | | | | | | | |
| | Avena sativa | | | | | | | | | | |
| | (oat) kernel | | | | | | | | | | |
| | extract | | | | | | | | | | |
| Dragosan W/O | Polyglyceryl-3 | | | | | | 1.0 | | | | |
| Liquid (Symrise) | polyricinoleate, | | | | | | | | | | |
| | sorbitan | | | | | | | | | | |
| | isostearate | | | | | | | | | | |
| Dragosan W/O P | Sorbitan | | | | | | 6.0 | | | | |
| (Symrise) | isostearate, | | | | | | | | | | |
| | hydrogenated | | | | | | | | | | |
| | castor oil, | | | | | | | | | | |
| | ceresin, | | | | | | | | | | |
| | beeswax (cera | | | | | | | | | | |
| | alba) | | | | | | | | | | |
| Dragoxat EH | Ethylhexyl | 3.0 | 3.0 | | 4.0 | | | | 3.0 | | |
| (Symrise) | ethylhexanoate | | | | | | | | | | |
| Dragoxat 89 | Ethylhexyl | | | | | | | | | 2.0 | |
| (Symrise) | ethylisono- | | | | | | | | | | |
| | nanoate | | | | | | | | | | |
| EDETA B powder | Tetrasodium | | | | | | | 0.1 | | | |
| (BASF) | EDTA | | | | | | | | | | |
| EDETA DB | Disodium EDTA | | | | | 0.1 | | | 0.1 | | |
| (BASF) | | | | | | | | | | | |
| Emulsiphos | Potassium cetyl | | 2.0 | | | 1.5 | | | | 2.0 | |
| (Symrise) | phosphate, | | | | | | | | | | |
| | hydrogenated | | | | | | | | | | |
| | palm glycerides | | | | | | | | | | |
| Ethanol 96% | Ethanol | | | | | | | | 2.0 | | 30.0 |
| Extrapone Green | Glycerin, water | | 0.2 | | | | | | | | |
| Tea GW (Symrise) | (aqua), Camellia | | | | | | | | | | |
| | sinensis leaf | | | | | | | | | | |
| | extract | | | | | | | | | | |
| Extrapone Witch | Propylene | | | | | | 1.0 | | | | |
| Hazel Distillate | glycol, | | | | | | | | | | |
| colorless | Hamamelis | | | | | | | | | | |
| (Symrise) | virginiana (witch | | | | | | | | | | |
| | hazel) water, | | | | | | | | | | |
| | water (aqua), | | | | | | | | | | |
| | Hamamelis | | | | | | | | | | |
| | virginiana (witch | | | | | | | | | | |
| | hazel) extract | | | | | | | | | | |
| Extrapone | Glycerin, water | | 0.3 | | | | | | | 0.5 | |
| Rosemary GW | (aqua), | | | | | | | | | | |
| (Symrise) | Rosmarinus | | | | | | | | | | |
| | officinalis | | | | | | | | | | |
| | (rosemary) leaf | | | | | | | | | | |
| | extract | | | | | | | | | | |
| Farnesol | Farnesol | | | | | | | | | | 0.5 |
| (Symrise) | | | | | | | | | | | |
| Frescolat ML | Menthyl lactate | | | 0.8 | | | | | | | |
| cryst. (Symrise) | | | | | | | | | | | |
| Genapol LRO | Sodium laureth | | | | | | | 37.0 | | | |
| liquid (Cognis) | sulfate | | | | | | | | | | |
| Givobio GZN | Zinc gluconate | | | | | | | | | 0.5 | |
| (Seppic) | | | | | | | | | | | |
| Glycerin 85% | Glycerin | 3.0 | 2.0 | 4.0 | | 4.7 | 2.0 | | 1.5 | 3.0 | |
| Hydrolite-5 | Pentylene glycol | | | | 5.0 | | | | 3.5 | | |
| (Symrise) | | | | | | | | | | | |
| Hydroviton | Water, glycerin, | | | | | | | | | 1.0 | |
| (Symrise) | sodium lactate, | | | | | | | | | | |
| | TEA lactate, | | | | | | | | | | |
| | serine, lactic | | | | | | | | | | |
| | acid, urea, | | | | | | | | | | |
| | sorbitol, sodium | | | | | | | | | | |
| | chloride, lauryl | | | | | | | | | | |
| | diethylene- | | | | | | | | | | |
| | diaminoglycine, | | | | | | | | | | |
| | lauryl amino- | | | | | | | | | | |
| | propylglycine, | | | | | | | | | | |
| | allantoin | | | | | | | | | | |
| Irgasan DP 300 | Triclosan | | | | | | | | | | 0.3 |
| (Ciba Gelgy) | | | | | | | | | | | |
| Isodragol | Triisononanoin | | 2.0 | | | | | | | 3.0 | |
| (Symrise) | | | | | | | | | | | |
| Isopropyl palmitate | Isopropyl | 4.0 | | | | | | | 4.0 | | |
| (Symrise) | palmitate | | | | | | | | | | |
| Karion F (Merck) | Sorbitol | | | | | | 2.0 | | | | |
| Keltrol RD (CP- | Xanthan gum | 0.2 | 0.1 | | | | | | | | |
| Kelco) | | | | | | | | | | | |
| Keltrol T (Danby- | Xanthan gum | | | | | 0.2 | | | 0.3 | | |
| Chemie) | | | | | | | | | | | |
| Kojic acid | Kojic acid | 1.0 | | | | | | | | | |
| (Cosmetochem) | | | | | | | | | | | |
| Lanette 16 | Cetyl alcohol | 1.0 | | | | | | | 1.0 | | |
| (Cognis) | | | | | | | | | | | |
| Lanette O | Cetearyl alcohol | | 3.0 | | | 1.0 | | | | 2.0 | |
| (Cognis) | | | | | | | | | | | |
| Lara Care A-200 | Galactoarabinan | | | 0.3 | | | | | | | |
| (Rahn) | | | | | | | | | | | |
| Magnesium | Magnesium | | | | | | 0.7 | | | | |
| chloride (Merck) | chloride | | | | | | | | | | |
| Merquat 550 | Polyquaternium-7 | | | | | | | 0.5 | | | |
| (Ondeo Nalco) | | | | | | | | | | | |
| NaOH 10% sol. | Sodium | | | | | | | | | 0.3 | |
| | hydroxide | | | | | | | | | | |
| Naringin (Exquim) | 4′,5,7-trihydroxy- | | | | | | | 0.5 | 2.0 | | |
| | flavone 7-O-neo- | | | | | | | | | | |
| | hesperidoside | | | | | | | | | | |
| Sodium benzoate | Sodium | | | | | | | 0.5 | | | |
| | benzoate | | | | | | | | | | |
| Natrosol 250 HHR | Hydroxyethyl- | | | | | | | | | | 0.3 |
| (Aqualon) | cellulose | | | | | | | | | | |
| Neo Heliopan 357 | Butyl methoxy- | | | | | 1.0 | | | | | |
| (Symrise) | dibenzoyl- | | | | | | | | | | |
| | methane | | | | | | | | | | |
| Neo Heliopan AP | Disodium phenyl | | | | | 10 | | | | | |
| (Symrise) | dibenzimidazole | | | | | | | | | | |
| (10% as sodium | tetrasulfonate | | | | | | | | | | |
| salt) | | | | | | | | | | | |
| Neo Heliopan AV | Ethylhexyl | | | | | 3.0 | | | | | |
| (Symrise) | methoxy- | | | | | | | | | | |
| | cinnamate | | | | | | | | | | |
| Neo Heliopan | Phenylbenz- | | | | | 6.7 | | | | | |
| Hydro (Symrise) | imidazole sulfonic acid | | | | | | | | | | |
| (15% as sodium | | | | | | | | | | | |
| salt) | | | | | | | | | | | |
| Neo Heliopan | 4-Methylbenzyl- | | | | | 1.5 | | | | | |
| MBC (Symrise) | idene camphor | | | | | | | | | | |
| Neo Heliopan OS | Ethylhexyl | | | | | 5.0 | | | | | |
| (Symrise) | salicylate | | | | | | | | | | |
| Neutral oil | Caprylic/capric | 6.0 | | | 4.0 | 2.0 | | | 6.0 | 10.0 | |
| | triglyceride | | | | | | | | | | |
| Oxynex 2004 | BHT | | | | | | 0.1 | | | | |
| (Merck) | | | | | | | | | | | |
| Paraffin oil 5 E | Paraffinum | | | | 4.0 | | | | | | |
| (Parafluid) | liquidum | | | | | | | | | | |
| PCL Liquid 100 | Cetearyl | 3.0 | 5.0 | | 7.0 | | | | | | |
| (Symrise) | ethylhexoate | | | | | | | | | | |
| PCL Solid | Stearyl | | 2.0 | | | | | | | | |
| (Symrise) | heptanoate, | | | | | | | | | | |
| | stearyl caprylate | | | | | | | | | | |
| PCL Liquid | Cetearyl | | | | | | 12.0 | | 3.0 | | |
| (Symrise) | ethylhexanoate, | | | | | | | | | | |
| | isopropyl | | | | | | | | | | |
| | myristate | | | | | | | | | | |
| Pemulen TR-2 | Acrylates/C10-30 | | | 0.3 | 0.2 | | | | | | |
| (Noveon) | alkyl acrylate | | | | | | | | | | |
| | crosspolymer | | | | | | | | | | |
| 4-(1-Phenylethyl)- | 4-(1-Phenyl- | 0.5 | | | | | | | | | |
| 1,3-benzenediol | ethyl)-1,3- | | | | | | | | | | |
| | benzenediol | | | | | | | | | | |
| 1,2-Propylene | Propylene glycol | | 5.0 | | | | | | | | |
| glycol 99P GC | | | | | | | | | | | |
| Pseudoceramide | N-(1-hexa- | | 0.1 | | | | | 0.2 | | 0.5 | |
| 391 | decanol)-4- | | | | | | | | | | |
| | hydroxy-L- | | | | | | | | | | |
| | proline (1- | | | | | | | | | | |
| | hexadecyl ester) | | | | | | | | | | |
| Retinyl palmitate | Retinyl palmitate | | | | | | 0.2 | | | | |
| in oil (DSM | | | | | | | | | | | |
| Nutritional | | | | | | | | | | | |
| Products) | | | | | | | | | | | |
| Sepigel 305 | Polyacrylamide, | | | | | | | | 1.0 | | |
| | C13-14 | | | | | | | | | | |
| | isoparaffin, | | | | | | | | | | |
| | laureth-7 | | | | | | | | | | |
| Sodium chloride | Sodium chloride | | | | | | | 1.0 | | | |
| Sodium hydroxide | Sodium | | 0.3 | 0.6 | 0.4 | | | | | | |
| (10% sol.) | hydroxide | | | | | | | | | | |
| Solubilizer 611674 | PEG-40 | | | | | | | | | | 2.0 |
| (Symrise) | hydrogenated | | | | | | | | | | |
| | castor oil, | | | | | | | | | | |
| | trideceth-9, | | | | | | | | | | |
| | water (aqua) | | | | | | | | | | |
| Sunflower oil | Helianthus | | | | | | 5.0 | | | | |
| (Wagner) | annuus | | | | | | | | | | |
| | (sunflower) seed | | | | | | | | | | |
| | oil | | | | | | | | | | |
| Sweet almond oil | Prunus dulcis | | | | | | 5.0 | | | | |
| (Wagner) | | | | | | | | | | | |
| Symdiol 66 | 1,2-Hexanediol, | 0.5 | | | | | | | | | |
| (Symrise) | caprylyl glycol | | | | | | | | | | |
| Symrise | Fragrance | 0.3 | 0.3 | 0.3 | 0.2 | 0.4 | 0.4 | 0.5 | 0.3 | 0.3 | 1.0 |
| Fragrance | | | | | | | | | | | |
| Tamasterol (Tama | Phytosterols | | | | | | | | | 0.3 | |
| Biochemicals) | | | | | | | | | | | |
| Tego Betaine L7 | Cocamidopropyl | | | | | | | 6.0 | | | |
| (Degussa) | betaine | | | | | | | | | | |
| Tegosoft PC 31 | | | | | | | | | | 0.3 | |
| (Degussa) | | | | | | | | | | | |
| Tegosoft TN | C12-15 alkyl | | | 5.0 | | 5.0 | | | | | |
| (Degussa) | benzoate | | | | | | | | | | |
| Triethanolamine, | Triethanolamine | | | | | 0.5 | | | | | |
| 99% | | | | | | | | | | | |
| Tocopherol | Tocopheryl | | | 0.5 | | 0.5 | 3.0 | | | 0.3 | |
| acetate (DSM | acetate | | | | | | | | | | |
| Nutritional | | | | | | | | | | | |
| Products) | | | | | | | | | | | |
| Zirkonal L 450 | Aluminium | | | | | | | | | | 37.0 |
| (BK Giulini) | zirconium | | | | | | | | | | |
| | pentachloro- | | | | | | | | | | |
| | hydrate | | | | | | | | | | |
| | (40% aqueous | | | | | | | | | | |
| | solution) | | | | | | | | | | |
| Water, | Water (aqua) | ad 100 | ad 100 | ad 100 | ad 100 | ad 100 | ad 100 | ad 100 | ad 100 | ad 100 | ad 100 |
| demineralized | | | | | | | | | | | |

Key to the following table
6.1 = Skin-lightening O/W day cream
6.2 = Skin-soothing O/W lotion with plant extracts
6.3 = Aftersun balm
6.4 = Body spray
6.5 = Sunscreen lotion (O/W), broadband protection
6.6 = W/O night cream
6.7 = Shampoo
6.8 = Self-tanning cream
6.9 = O/W barrier repair cream
6.10 = Antiperspirant/deodorant roll-on

1. A ternary mixture containing, substantially consisting of or consisting of
a) an extract of Aloe plant material,
b) ascorbic acid and/or the derivative(s) thereof, preferably at least one solvate and/or salt of ascorbic acid and/or at least one ascorbic acid prodrug, and
c) an extract of rosaceous plant material, except for an aftersun balm simultaneously containing 3.0 wt. % of an Aloe barbadensis leaf extract 10/1 in water, 1.0 wt. % of sodium ascorbyl phosphate and 0.5 wt. % of maltodextrin Rubus fruticosus leaf extract, in each case relative to the entire balsam.

2. The mixture as claimed in claim 1, containing
a) an extract of Aloe plant material,
b) sodium and/or magnesium ascorbyl phosphate, and
c) an extract selected from the group consisting of blackberry leaf extract, raspberry leaf extract, strawberry leaf extract and meadowsweet herb extract, or two or more such extracts.

3. The mixture as claimed in claim 1 or claim 2, wherein the proportions of the components in each case amount to:
component a): 33-99 wt. %, preferably 80-98 wt. % and particularly preferably 89-96.51 wt. %;
component b): 0.99-32.99 wt. %, preferably 1.9-15 wt. % and particularly preferably 3.22-9 wt. %;
component c): 0.01-10 wt. %, preferably 0.1-4 wt. % and particularly preferably 0.27-2 wt. %; in each case relative to the total of components a), b) and c).

4. The mixture as claimed in claims 1, 2 or 3, wherein the proportions the component in each case amount to:
component a): 85-92 wt. %, preferably 88-92 wt. %, particularly preferably 90 wt. %,
component b): 7-10 wt. %, preferably 9 wt. %,
component c): 0.5-2 wt. %, preferably 0.8-1.2 wt. %, particularly preferably 1 wt. %, in each case relative to the total of components a), b) and c), with component b) consisting of magnesium ascorbyl phosphate and component c) of raspberry leaf extract.

5. A cosmetic, pharmaceutical and/or oral hygiene preparation, containing a mixture as claimed in any one of claims 1, 2, 3 or 4, wherein the concentration of the mixture amounts to 0.001-20 wt. %, preferably 0.01-10 wt. % and particularly preferably amounts to 0.1-5 wt. %, relative to the entire preparation.

6. A cosmetic, pharmaceutical and/or oral hygiene preparation as claimed in claim 5, containing the mixture as claimed in any one of claims 1, 2, 3 or 4 in a concentration sufficient
for stimulating collagen synthesis and/or
for slowing down skin ageing and/or
for preventing and/or slowing down periodontitis and/or caries.

7. A method for manufacturing a pulverulent mixture as claimed in claims 1, 2, 3 or 4, comprising mixing components a), b) and c) in their respective selected amounts to obtain a homogeneous pulverulent mixture.

8. A method for manufacturing a liquid mixture as claimed in claims 1, 2, 3 or 4, comprising
(i) mixing components a), b) and c) in their respective selected amounts to obtain a, preferably homogeneous, pulveruient mixture, and subsequently
(ii) dissolving the resultant pulverulent mixture in a medium containing
water and/or
an alcohol selected from the group consisting of methanol, ethanol, n-propanol, isopropanol, and/or
an aliphatic polyol selected from the group consisting of glycerin, propylene glycol, butylene glycol, 1,2-alkanediols having 3-10 carbon atoms.

9. Use of a mixture as claimed in any one of claims 1, 2, 3 or 4 or of a cosmetic, pharmaceutical and/or oral hygiene preparation as claimed in claim 5 or claim 6
for stimulating collagen synthesis in non-therapeutic manner and/or
for slowing down skin ageing in non-therapeutic manner and/or
for preventing and/or slowing down periodontitis and/or caries in non-therapeutic manner.
